(19) United States
(12) Patent Application Publication
(10) Pub. No.: US 2025/0143997 A1
(43) Pub. Date: May 8, 2025

(54) Personal Care Compositions

(71) Applicant: Colgate-Palmolive Company, New York, NY (US)

(72) Inventors: Samuel MCANGEL, Princeton, NJ (US); Ningwei LI, North Brunswick, NJ (US); Jonghun LEE, Metuchen, NJ (US)

(73) Assignee: Colgate-Palmolive Company, New York, NY (US)

(21) Appl. No.: 18/715,218

(22) PCT Filed: Dec. 2, 2022
(86) PCT No.: PCT/US2022/051687

§ 371 Date: May 31, 2024

Related U.S. Application Data
(60) Provisional application No. 63/285,308, filed on Dec. 2, 2021.

(51) Int. Cl. A61K 8/64; A45D 34/00; A61K 8/04; A61K 8/46; A61K 8/60; A61Q 5/02; A61Q 19/10

(52) U.S. Cl. CPC A61K 8/645; A45D 34/00; A61K 8/046; A61K 8/463; A61K 8/602; A61K 8/64; A61Q 5/02; A61Q 19/10; A45D 2200/055

(57) ABSTRACT

Disclosed are personal care compositions, e.g., foaming facial cleanser, the contain a polyol and a surfactant system that comprises: an amount of an anionic surfactant, wherein the surfactant is an anionic derivative of proteins of plant origin or of silk proteins (e.g., sodium cocoyl apple amino acid surfactant); and an amount of a non-ionic surfactant selected from an alkyl glucoside, a lactylate having carbon chains between C₈ and C₁₆, and a combination of two or more thereof, as well as to methods of using these compositions.

### CROSS-REFERENCE TO RELATED APPLICATION

This application claims the benefit of priority from U.S. Provisional Application No. 63/285,308, entitled “Personal Care Compositions” and filed Dec. 2, 2021, the contents of which is hereby incorporated herein in its entirety.

### BACKGROUND

Liquid cleansing compositions, such as aqueous foaming compositions like hand soaps, facial soaps or cleansers, body washes, and shower gels, should desirably have acceptable levels of foam production for consumer satisfaction. Foam production, for example, is a key attribute for consumers when choosing products. To achieve a desirable foam production, manufacturers incorporate sufficient quantities of foaming agents, such as surfactants, into the compositions.

However, in recent years, consumer demand for more natural products has increased—e.g., interest in the origin of raw materials and products with ingredients with a high degree of naturality. It is a challenge to produce formulations that have the physical characteristics that consumers expect, including desirable amounts of foam production and sufficient viscosity, while simultaneously achieving a high degree of naturality. Moreover, liquid consumer products normally contain different species of surfactants. Consequently, manufacturers are shifting to the use of natural/sustainable surfactant substitutes. One challenge with surfactant substitutes, however, is maintaining an acceptable foaming volume that is still acceptable for consumers.

Accordingly, there is an ongoing need for improved a personal care products that meet the desires of consumers.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

The personal care compositions of the instant disclosure surprisingly achieve a high level of foaming quality, e.g., foaming volume with a reduced amount or no sulfate-based anionic surfactants (e.g., sodium lauryl sulfate). Without being bound by theory, sulfate systems are typically recognized as being easier to work with because they provide a high level of foam and can facilitate thickening with salt. It was discovered that personal care compositions comprising certain surfactant systems are able to achieve a desirably high level of level of foam production, provide a gentle cleansing (e.g., does not strip keratin substrates and/or overly dries keratin substrates, such as skin.), while utilizing a minimal amount or being free of sulfate-based surfactants. Additionally or alternatively, certain embodiments of the personal care compositions achieve the foregoing benefits while having a high level of naturality (e.g., have a high percent of ingredients that are natural and/or naturally derived). In one aspect, the personal care compositions of the disclosure can comprise a naturally derived oil while maintaining the clarity that is sometimes lost with the inclusion of oils. In another aspect, the personal care compositions of the disclosure comprises one or more actives that can provide improved hydration and soothing to the skin, e.g., wherein the skin barrier is damaged and/or irritated.

In one aspect, the disclosure provides a personal care composition (e.g., a skin care composition) (e.g., a foaming facial cleanser) comprising:
an effective amount of a polyol emollient (e.g., glycerine, 1,3-propanediol, isoprene glycol, pentylene glycol, hexylene glycol, glycols such as ethylene glycol, propylene glycol, butylene glycol, diethylene glycol and dipropylene glycol, polyglycerols with 2 to 6 repeating units, for instance diglycerol, erythritol, arabitol, adonitol, sorbitol, dulcitol, glucose, fructose, xylose, trehalose, sucrose, maltose, saccharose and lactose, and mixtures thereof.)
a surfactant system, wherein the surfactant system comprises:
   an effective amount of an anionic surfactant, wherein the surfactant is an anionic derivative of proteins of plant origin or of silk proteins (e.g., sodium cocoyl apple amino acid surfactant); and
   an effective amount of a non-ionic surfactant, (e.g., wherein the non-ionic surfactant is selected from an alkyl glucoside, a lactylate having carbon chains between C₈ and C₁₆, and a blend of an alkyl glucoside and a lactylate having carbon chains between C₈ and C₁₆ (e.g., ammonium, sodium, potassium and alkanolamine (e.g., triethanolamine) salts of lauroyl lactylate (e.g., sodium lauroyl lactylate), cocoyl lactylate, lauroyl lactylate, and caproyl lactylate); and
water.

In another aspect, the disclosure provides a method of depositing a topically active compound on the skin, comprising applying an effective amount of any of personal care compositions as disclosed herein to the skin.

Further areas of applicability of the present disclosure will become apparent from the detailed description provided hereinafter. It should be understood that the detailed description and specific examples, while indicating some typical aspects of the disclosure, are intended for purposes of illustration only and are not intended to limit the scope of the disclosure.

### BRIEF DESCRIPTION OF THE DRAWINGS

The features, and advantages of the invention will be apparent from the following more detailed description of certain embodiments of the invention and as illustrated in the accompanying drawings in which:

FIG. 1 is a graph of the average rheological foam curves for two non-limiting example compositions in accordance with aspects of the invention.

FIGS. 2A and 2B are bar graphs of the elasticity and yield stress, respectively, of the foam of two non-limiting example compositions in accordance with aspects of the invention.

FIG. 3 is a bar graph illustrating the amount of sebum removed by non-limiting example compositions or a control in accordance with aspects of the invention.

It should be understood that the various aspects are not limited to the compositions, arrangements, and instrumentality shown in the figures.

### DETAILED DESCRIPTION

The following description of various typical aspect(s) is merely exemplary in nature and is in no way intended to limit the disclosure, its application, or uses.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc. The term “about” when referring to a number means any number within a range of 10% of the number. For example, the phrase “about 2 wt. %” refers to a number between and including 1.8 wt. % and 2.2 wt. %.

As used herein, the term “effective amount” means the quantity of a surfactant required to provide adequate foam volume and structure to the personal care compositions. In one aspect, the “effective amount” of surfactant is the amount of surfactant required so that the foaming volume of the personal care composition is at parity, or improved, relative to a control composition (e.g., where the control contains a sulfate based anionic surfactant (e.g., sodium lauryl sulfate or sodium laureth sulfate)).

As used herein, “polyol” is understood to mean any organic molecule comprising at least two free hydroxyl groups.

Unless otherwise specified, all percentages and amounts expressed herein and elsewhere in the specification should be understood to refer to percentages based on the weight of the total composition. The amounts given are based on the active weight of the material.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

“Volatile”, as used herein, means having a flash point of less than about 100° C. “Non-volatile”, as used herein, means having a flash point of greater than about 100° C.

When referring to chemical structures, and names, the symbols “C”, “H”, and “O” mean carbon, hydrogen, and oxygen, respectively. The symbols “—”, “═” and “≡” mean single bond, double bond, and triple bond, respectively. For readability purposes, the chemical functional groups are in their adjective form; for each of the adjectives, the word “group” is assumed. For example, the adjective “alkyl” without a noun thereafter, should be read as “an alkyl group.”

Any member in a list of species that is used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that defines or exemplifies the genus is open, and it is given that other species may exist that define or exemplify the genus just as well as, or better than, any other species listed.

The phrases, “a mixture thereof,” “a combination thereof,” or “a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the personal care compositions of the instant disclosure can be free or essentially free of all components and elements positively recited throughout the instant disclosure. In some instances, the personal care compositions of the present disclosure may be substantially free of non-incidental amounts of the ingredient(s) or compound(s) described herein. A non-incidental amount of an ingredient or compound is the amount of that ingredient or compound that is added into the personal care composition by itself. For example, a personal care composition may be substantially free of a non-incidental amount of an ingredient or compound, although such ingredient(s) or compound(s) may be present as part of a raw material that is included as a blend of two or more compounds.

The present invention provides, in an aspect, a personal care composition (Composition 1.0), (e.g., a foaming facial cleanser), comprising:
an effective amount of a polyol (e.g., glycerin, 1,3-propanediol, isoprene glycol, pentylene glycol, hexylene glycol, glycols such as ethylene glycol, propylene glycol, butylene glycol, diethylene glycol and dipropylene glycol, polyglycerols with 2 to 6 repeating units, for instance diglycerol, erythritol, arabitol, adonitol, sorbitol, dulcitol, glucose, fructose, xylose, trehalose, sucrose, maltose, saccharose and lactose, or mixtures of two or more thereof.);
a surfactant system, wherein the surfactant system comprises:
   a. an effective amount of an amphoteric surfactant (e.g., a sultaine amphoteric surfactant, betaine surfactant, etc.),
   b. an effective amount of an anionic surfactant, wherein the surfactant is an anionic derivative of proteins of plant origin or of silk proteins (e.g., sodium cocoyl apple amino acid surfactant), and
   c. an effective amount of a non-ionic surfactant, preferably selected from an alkyl glucoside, a lactylate having carbon chains between C₈ and C₁₆, and a combination of two or more thereof (e.g., wherein the non-ionic surfactant is a blend of an alkyl glucoside and a lactylate having carbon chains between C₈ and C₁₆ (e.g., ammonium, sodium, potassium and alkanolamine (e.g., triethanolamine) salts of lauroyl lactylate (e.g., sodium lauroyl lactylate), cocoyl lactylate, lauroyl lactylate, and caproyl lactylate)); and
water.

For example, aspects of the invention are directed to:
1.1. Personal Care Composition 1.0, wherein the amphoteric surfactant is present in an amount from 0.1 to 15 wt. % of the total composition. In some instances, the total amount of amphoteric surfactant in the personal care composition is from 0.1 to 15 wt. %, 0.1 to about 12 wt. %, 0.1 to about 10 wt. %, 0.1 to about 8 wt. %, 0.1 to about 6 wt. %, 0.1 to about 4 wt. %, 0.1 to about 3 wt. %, 0.1 to about 2 wt. %; from about 1 to 15 wt. %, about 1 to about 12 wt. %, about 1 to about 10 wt. %, about 1 to about 8 wt. %, about 1 to about 6 wt. %, about 1 to about 4 wt. %, about 1 to about 3 wt. %, about 1 to about 2 wt. %; from about 2 to 15 wt. %, about 2 to about 12 wt. %, about 2 to about 10 wt. %, about 2 to about 8 wt. %, about 2 to about 6 wt. %, about 2 to about 4 wt. %; from about 4 to 15 wt. %, about 4 to about 12 wt. %, about 4 to about 10 wt. %, about 4 to about 8 wt. %, about 4 to about 6 wt. %; from about 6 to 15 wt. %, about 6 to about 12 wt. %, about 6 to about 10 wt. %, about 6 to about 8 wt. %; from about 8 to 15 wt. %, about 8 to about 12 wt. %, about 8 to about 10 wt. %; from about 10 to 15 wt. %, about 10 to about 12 wt. %, about 12 to 15 wt. %, or any range or subrange thereof, based on the total weight of the personal care composition. The personal care composition may, in some instances, comprise a sultaine surfactant e.g., from 0.1 to 15 wt. % based on the weight of the total composition) (e.g., 0.5 to 7 wt. % based on the weight of the total composition) (e.g., about 5 wt. % based on the weight of the total composition).
1.2. The preceding personal care composition, wherein the sultaine surfactant (e.g., a hydroxysultaine) is an alkyl sultaines having from 6-20 carbons, 8-18 carbons, 8-16 carbons, or 10-16 carbons. Non-limiting examples of alkyl sultaines include hydroxyl sultaines having a structure according to the following formula:

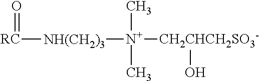

wherein R is an alkyl group having 8-18 carbon atoms. Examples of alkyl sultaine surfactants include, but are not limited to, cocamidopropyl hydroxysultaine, lauryl hydroxysultaine, and a combination of two or more thereof. In some embodiments, the sultaine surfactant is selected from the group consisting of: lauramidopropyl hydroxysultaine, cocamidopropyl hydroxysultaine, oleamidopropyl hydroxysultaine, tallowamidopropyl hydroxysultaine, erucamidopropyl hydroxysultaine, lauryl hydroxysultaine, lauramidopropyl hydroxysultaine, and combinations thereof.
1.3. The preceding personal care composition, wherein the sultaine amphoteric surfactant comprises cocamidopropyl hydroxysultaine.
1.4. Any foregoing personal care compositions, wherein the amphoteric surfactant is or comprises a betaine surfactant (e.g., from 0.1 to 15 wt. % based on the weight of the total composition) (e.g., 0.5 to 4 wt. % based on the weight of the total composition) (e.g., about 3 wt. % based on the weight of the total composition) (e.g., about 4 wt. % based on the weight of the total composition). The betaine surfactant may be chosen from alkyl betaines, such as coco dimethyl carboxymethyl betaine, lauryl dimethyl carboxy-methyl betaine, lauryl dimethyl alpha-carboxyethyl betaine, cetyl dimethyl carboxymethyl betaine, lauryl bis-(2-hydroxyethyl)carboxy methyl betaine, stearyl bis-(2-hydroxypropyl)carboxymethyl betaine, oleyl dimethyl gamma-carboxypropyl betaine, lauryl bis-(2-hydroxypropyl)alpha-carboxyethyl betaine; sulfobetaines, such as coco dimethyl sulfopropyl betaine, stearyl dimethyl sulfopropyl betaine, amido betaines, amidosulfobetaines and a combination of two or more thereof. The betaine surfactant may be selected from coca betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, and combinations of two or more thereof. In some embodiments, the betaine surfactant is selected from coco betaine, cocamidopropyl betaine, behenyl betaine, capryl/capramidopropyl betaine, lauryl betaine, and a combination of two or more thereof.
1.5. The preceding personal care composition, wherein the betaine surfactant is a C₈-C₁₆ aminopropyl betaine (e.g., cocamidopropyl betaine).
1.6. The preceding personal care composition wherein the C₈-C₁₆ aminopropyl betaine is cocamidopropyl betaine.
1.7. The preceding personal care composition wherein the cocamidopropyl betaine, is present in an amount of from 0.5 to 5 wt. % based on the weight of the total composition.
1.8. The preceding personal care composition, wherein the cocamidopropyl betaine is from 1 to 5 wt. % based on the weight of the total composition.
1.9. Any of the preceding personal care compositions, wherein the total amount of surfactants present in the composition is from 5 to 30 wt. %, (e.g., from 5 to 15 wt. %), based on the weight of the personal care composition. For instance, the total amount of surfactant(s) in the personal care composition may be from 5 to about 30 wt. %, 5 to about 28 wt. %, 5 to about 26 wt. %, 5 to about 24 wt. %, 5 to about 22 wt. %, 5 to about 20 wt. %, 5 to about 18 wt. %, 5 to about 15 wt. %, 5 to about 12 wt. %, 5 to about 9 wt. %; from about 8 to about 30 wt. %, about 8 to about 28 wt. %, about 8 to about 26 wt. %, about 8 to about 24 wt. %, about 8 to about 22 wt. %, about 8 to about 20 wt. %, about 8 to about 18 wt. %, about 8 to about 16 wt. %, about 8 to about 14 wt. %, about 8 to about 12 wt. %; from about 10 to about 30 wt. %, about 10 to about 28 wt. %, about 10 to about 26 wt. %, about 10 to about 24 wt. %, about 10 to about 22 wt. %, about 10 to about 20 wt. %, about 10 to about 18 wt. %, about 10 to about 16 wt. %, about 10 to about 14 wt. %; from about 12 to about 30 wt. %, about 12 to about 28 wt. %, about 12 to about 26 wt. %, about 12 to about 24 wt. %, about 12 to about 22 wt. %, about 12 to about 20 wt. %, about 12 to about 18 wt. %, about 12 to about 16 wt. %; from about 14 to about 30 wt. %, about 14 to about 28 wt. %, about 14 to about 26 wt. %, about 14 to about 24 wt. %, about 14 to about 22 wt. %, about 14 to about 20 wt. %, about 14 to about 18 wt. %; from about 16 to about 30 wt. %, about 16 to about 28 wt. %, about 16 to about 26 wt. %, about 16 to about 24 wt. %, about 16 to about 22 wt. %, about 16 to about 20 wt. %; from about 18 to about 30 wt. %, about 18 to about 28 wt. %, about 18 to about 26 wt. %, about 18 to about 24 wt. %, about 18 to about 22 wt. %; from about 20 to about 30 wt. %, about 20 to about 28 wt. %, about 20 to about 26 wt. %, about 20 to about 24 wt. %; from about 22 to about 30 wt. %, about 22 to about 28 wt. %, about 22 to about 26 wt. %, about 22 to about 24 wt. %, or any range or subrange thereof, based on the total weight of the personal care composition.
1.10. Any of the preceding personal care compositions, wherein the anionic surfactant comprises an anionic derivative of a protein hydrolysate of plant origin, and wherein the protein hydrolysate comprises a hydrophobic group (e.g., wherein the hydrophobic group is a fatty chain, e.g., an alkyl chain comprising from 10 to 22 carbon atoms) (e.g., from 1 to 10 wt. % based on the weight of the personal care composition) (e.g., about 5 wt. % based on the weight of the personal care composition). The personal care composition may include the anionic surfactant(s) in an amount that may vary, but typically in an amount from 1 to 10 wt. %, based on the total weight of the personal care composition. For example, the amount of anionic surfactants present in the personal care composition may be from 1to 10 wt. %, 1 to about 8 wt. %, 1 to about 6 wt. %, 1 to about 4 wt. %, 1 to about 3 wt. %, 1 to about 2 wt. %; from about 2 to 10 wt. %, about 2 to about 8 wt. %, about 2 to about 6 wt. %, about 2 to about 4 wt. %; from about 4 to 10 wt. %, about 4 to about 8 wt. %, about 4 to about 6 wt. %; about 6 to 10 wt. %, about 6 to about 8 wt. %, about 8 to 10 wt. %, or any range or subrange thereof, based on the total weight of the personal care composition.
1.11. Any of the preceding personal care compositions, wherein the anionic surfactant is an anionic derivatives of proteins of plant origin are protein hydrolysates containing a hydrophobic group, it being possible for the said hydrophobic group to be naturally present in the protein or to be added by reaction of the protein and/or of the protein hydrolysate with a hydrophobic compound. The proteins are of plant origin or are derived from silk, and the hydrophobic group may be a fatty chain, such as an alkyl chain comprising from 10 to 22 carbon atoms.
1.12. Any of the preceding personal care compositions, where the anionic derivatives of hydrolysates proteins of plant origin are selected from those made from apple, wheat, soybean or oat protein hydrolysates comprising an alkyl chain having from 10 to 22 carbon atoms, and salts thereof. The alkyl chain may, in some preferred embodiments, be a lauryl chain and the cationic group may be a sodium, potassium and/or ammonium.
1.13. Any of the preceding personal care compositions, where the protein hydrolysates comprise a hydrophobic group. In some embodiments, the anionic derivatives of hydrolysates proteins may include, e.g., salts of protein hydrolysates where the protein is a silk protein modified by lauric acid; salts of protein hydrolysates where the protein is a wheat protein modified by lauric acid, such as potassium lauroyl wheat amino acids and sodium lauroyl wheat amino acids; salts of protein hydrolysates where the protein is an oat protein comprising an alkyl chain having from 10 to 22 carbon atoms and especially salts of protein hydrolysates where the protein is an oat protein modified by lauric acid, such as sodium lauroyl oat amino acids; or salts of apple protein hydrolysates comprising an alkyl chain having from 10 to 22 carbon atoms, such as sodium cocoyl apple amino acids. In some embodiments, the anionic derivatives of proteins of plant origin are protein hydrolysates are formed from a mixture of lauroyl amino acids (aspartic acid, glutamic acid, glycine, alanine) neutralized with sodium N-methylglycinate, such as sodium cocoyl amino acids, and sodium cocoyl glycinate.
1.14. The preceding composition, wherein the protein hydrolysate is an apple, wheat, soybean or oat protein hydrolysate comprising an alkyl chain having from 10 to 22 carbon atoms, and salts thereof.
1.15. The preceding composition, wherein the protein hydrolysate is an apple protein hydrolysate comprising an alkyl chain having from 10 to 22 carbon atoms, (e.g., a sodium salt sold under the name Proteol APL (30% aqueous glycol solution) from SEPPIC (CTFA name: sodium cocoyl apple amino acids)). Examples of anionic derivative of a protein hydrolysate of plant origin include potassium undecylenoyl hydrolyzed soy protein, sodium undecylenoyl hydrolyzed soy protein, sodium dilauramidoglutamide lysine, and, optionally, sodium cocoyl threoninate, sodium cocoyl glutamate, disodium cocoyl glutamate, and a combination of two or more thereof.
1.16. The preceding composition, wherein the anionic surfactant comprises sodium cocoyl apple amino acids (e.g., from 1%-10% by wt.) (e.g., about 5% by wt.).
1.17. Any of the preceding personal care compositions, wherein the anionic surfactants further comprises an acyl amino acid surfactant. The acyl amino acid may be chosen from amino acid surfactants based on alanine, arginine, aspartic acid, glutamic acid, glycine, isoleucine, leucine, lysine, phenylalanine, serine, tyrosine, valine, sarcosine, threonine, and taurine. The cation associated with the acyl amino acid can be sodium or potassium. Alternatively, the cation can be an organic salt such as triethanolamine (TEA) or a metal salt. In some embodiments, the anionic surfactant may further comprise acyl taurates, acyl glycinates (e.g., sodium cocoyl glycinate), acyl glutamates, and a combination thereof. The alkyl group of the acyl amino acid surfactant may comprise from 8 to 20 carbon atoms, 8 to 16 carbon atoms, 10 to 16 carbon atoms, or 10 to 13 carbon atoms.
1.18. Any of the preceding personal care compositions, wherein the anionic surfactants further comprises an acyl taurate. Examples of acyl taurates include those of the following formula:

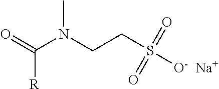

wherein R, R¹, R² and R³ are each independently selected from H or an alkyl chain having 1-24 carbon atoms, or from 6-20 carbon atoms, or from 8 to 16 carbon atoms, said chain being saturated or unsaturated, linear or branched, and X is COO⁻ or SO₃⁻. Non-limiting examples of acyl taurate salts include sodium cocoyl taurate, sodium methyl cocoyl taurate, oleoyl taurate, and combinations thereof. In at least one preferred embodiment, the personal care composition includes one or more anionic surfactant comprising sodium n-coconut acid-n-methyl taurate.
1.19. Any of the preceding personal care compositions, wherein the nonionic surfactant(s) is present in the personal care composition in an amount from about 1 to about 20 wt. %, based on the total weight of the personal care composition. For example, the total amount of nonionic surfactant(s) in the personal care composition may be from about 1 to about 20 wt. %, about 1 to about 18 wt. %, about 1 to about 16 wt. %, about 1 to about 14 wt. %, about 1 to about 12 wt. %, about 1 to about 10 wt. %, about 1 to about 8 wt. %, about 1 to about 7 wt. %, about 1 to about 6 wt. %, about 1 to about 5 wt. %; from about 3 to about 20 wt. %, about 3 to about 18 wt. %, about 3 to about 16 wt. %, about 3 to about 14 wt. %, about 3 to about 12 wt. %, about 3 to about 10 wt. %, about 3 to about 8 wt. %, about 3 to about 7 wt. %, about 3 to about 6 wt. %, about 3 to about 5 wt. %; from about 5 to about 20 wt. %, about 5 to about 18 wt. %, about 5 to about 16 wt. %, about 5 to about 14 wt. %, about 5 to about 12 wt. %, about 5 to about 10 wt. %, about 5 to about 8 wt. %; from about 7 to about 20 wt. %, about 7 to about 18 wt. %, about 7 to about 16 wt. %, about 7 to about 14 wt. %, about 7 to about 12 wt. %, about 7 to about 10 wt. %; from about 9 to about 20 wt. %, about 9 to about 18 wt. %, about 9 to about 16 wt. %, about 9 to about 14 wt. %, about 9 to about 12 wt. %; from about 11 to about 20 wt. %, about 11 to about 18 wt. %, about 11 to about 16 wt. %, about 11 to about 13 wt. %; from about 13 to about 20 wt. %, or about 13 to about 18 wt. %, including ranges and subranges thereof, based on the total weight of the personal care composition.
1.20. Any of the preceding personal care compositions, wherein the alkyl glucoside is C₆₋₂₅ alkyl glucoside, e.g., C₈₋₁₈ alkyl glucoside, C₁₀₋₁₈ alkyl glucoside or C₁₀₋₁₆ alkyl glucoside, optionally wherein the alkyl glucoside is selected from decyl glucoside, caprylyl/capryl glucoside, lauryl glucoside, coco-glucoside, octyl glucoside, cetearyl glucoside, cetyl glucoside, hexadecyl glucoside, arachidyl glucoside, and a combination thereof.
1.21. Any of the preceding personal care compositions, wherein the alkyl glucoside is selected from decyl glucoside, caprylyl/capryl glucoside, caprylyl glucoside, lauryl glucoside, coco-glucoside, octyl glucoside, cetearyl glucoside, cetyl glucoside, hexadecyl glucoside, arachidyl glucoside, and a combination thereof, (e.g., the alkyl glucoside is present in an amount of from 1 to 10 wt. %, from 2 to 9 wt. % based on the weight of the personal care composition. (e.g., about 8 wt. % based on the weight of the personal composition).
1.22. Any of the preceding personal care compositions, wherein the alkyl glucoside comprises decyl glucoside (e.g., from 1-10 wt. % based on the weight of the personal care composition) (e.g., about 5 wt. % based on the weight of the personal care composition).
1.23. Any of the preceding personal care compositions, wherein the alkyl glucoside comprises caprylyl/capryl glucoside (e.g., from 1-5 wt. % based on the weight of the personal care composition) (e.g., about 3 wt. % based on the weight of the personal are composition).
1.24. Any of the preceding personal care compositions, wherein the alkyl glucoside comprises a combination of decyl glucoside and caprylyl/capryl glucoside ((e.g., wherein the total combination is from 1 to 10 wt. % based on the weight of the personal care composition) (e.g., about 8 wt. % based on the weight of the personal care composition).
1.25. The preceding composition, wherein alkyl glucoside comprises decyl glucoside.
1.26. Any of the preceding personal care compositions, wherein the one or more alkyl glucoside(s) is present in an amount of from 0.5 to 15 wt. %, e.g., from about 1 to about 10 wt. %, from about 2 to about 9 wt. % (e.g., about 8 wt. %), based on the weight of the personal composition, optionally wherein the alkyl glucoside is present in an amount of from about 1.5 to about 8 wt. %, from about 1.5 to about 7 wt. %, or from about 1.5 to about 6.5 wt. %, based on the weight of the personal composition. In some instances, the total amount of alkyl glucoside(s) surfactant in the personal care composition is from 0.1 to 15 wt. %, 0.1 to about 12 wt. %, 0.1 to about 10 wt. %, 0.1 to about 8 wt. %, 0.1 to about 6 wt. %, 0.1 to about 4 wt. %, 0.1 to about 3 wt. %, 0.1 to about 2 wt. %; from about 1 to 15 wt. %, about 1 to about 12 wt. %, about 1 to about 10 wt. %, about 1 to about 8 wt. %, about 1 to about 6 wt. %, about 1 to about 4 wt. %, about 1 to about 3 wt. %, about 1 to about 2 wt. %; from about 2 to 15 wt. %, about 2 to about 12 wt. %, about 2 to about 10 wt. %, about 2 to about 8 wt. %, about 2 to about 6 wt. %, about 2 to about 4 wt. %; from about 4 to 15 wt. %, about 4 to about 12 wt. %, about 4 to about 10 wt. %, about 4 to about 8 wt. %, about 4 to about 6 wt. %; from about 6 to 15 wt. %, about 6 to about 12 wt. %, about 6 to about 10 wt. %, about 6 to about 8 wt. %; from about 8 to 15 wt. %, about 8 to about 12 wt. %, about 8 to about 10 wt. %; from about 10 to 15 wt. %, about 10 to about 12 wt. %, about 12 to 15 wt. %, or any range or subrange thereof, based on the total weight of the personal care composition.
1.27. Any of the preceding compositions, wherein the non-ionic surfactant is a lactylate surfactant having carbon chains between C₈ and C₁₆, such as sodium lauroyl (e.g., wherein the lactylate surfactant is present from about 1 to about 5 wt. % based on the weight of the personal composition) (e.g., about 2 wt. % based on the weight of the personal care composition). For example, in some embodiments, the amount of lactylate surfactant present in the personal care composition is from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; about 3 to about 5 wt. %, about 3 to about 4 wt. %, or any range or subrange thereof, based on the total weight of the personal care composition.
1.28. Any of the preceding compositions, wherein the non-ionic surfactant comprises a lactylate selected from sodium decanoyl/dodecanoyl lactylate, tea-lauroyl lactylate, sodium hexanoyl lactylate, sodium behenoyl lactylate, sodium oleoyl lactylate, sodium decanoyl lactylate, sodium cocoyl lactylate, sodium lauroyl lactylate, sodium isostearoyl lactylate, calcium stearoyl lactylate, sodium stearoyl lactylate, and a combination of two or more thereof.
1.29. The preceding composition, wherein alkyl glucoside comprises decyl glucoside and the lactylate comprises sodium lauroyl lactylate.
1.30. Any of the preceding personal care compositions, wherein the non-ionic surfactant is a blend of an alkyl glucoside and a lactylate having carbon chains between C₈ and C₁₆ and wherein the lactylate is selected from the group consisting of: ammonium, sodium, potassium and alkanolamine (e.g., triethanolamine) salts of lauroyl lactylate (e.g., sodium lauroyl lactylate), cocoyl lactylate, lauroyl lactylate, and caproyl lactylate) (e.g., wherein the surfactant is present from 1%-5% by wt. of the composition). The blend alkyl glucoside and a lactylate surfactants may include any of the alkyl glucosides and any of the lactylate surfactants disclosed herein.
1.31. Any of the preceding compositions, wherein the non-ionic surfactant is a blend of an alkyl glucoside and a lactylate having carbon chains between C₈ and C₁₆, and wherein the surfactant is a blend of decyl glucoside and sodium lauroyl lactylate (e.g., PUREACT 138) (e.g., wherein the surfactant is present from e.g., wherein the surfactant is present from 1%-5% by wt. of the composition) (e.g., about 2% by wt.).

1.32. Any of the preceding personal care compositions, in some embodiments, wherein the composition does not contain any surfactant other than the surfactants in the surfactant system.

1.33. Any of the preceding personal care compositions, wherein the composition comprises an oil selected from: meadowfoam oil, sunflower seed oil, olive oil, shea butter, jojoba oil, almond oil, grape seed oil, rose hip seed oil, mink oil, castor oil, soybean oil, mineral oil, and a combination thereof. The term “oil” means any fatty substance that is in liquid form at room temperature (24-25° C.) and at atmospheric pressure (760 mmHg). The oil may be a hydrocarbon-based oil, such as a saturated hydrocarbon oil, an unsaturated hydrocarbon oil, lipids, triglycerides, a natural oil, and/or a synthetic oil. The one or more oil(s) may be synthetic oils and/or plant based oils. For instance, the oil may be a synthetic oil, such as a mineral oil and/or an alkane (paraffins). Light (low viscosity) highly branched hydrocarbon oils may also be suitable in some instances. In some embodiments, the oil is chosen from plant based or vegetable based oils, such as coconut oil, soybean oil, canola oil, rapeseed oil, corn oil, cottonseed oil, olive oil, palm oil, peanut oil, safflower oil, sesame oil, sunflower oil, linseed oil, palm kernel oil, tung oil, jatropha oil, mustard oil, camelina oil, pennycress oil, castor oil, wheatgerm oil, apricot kernel oil, pistachio oil, poppy oil, pine oil, avocado oil, hazel nut oil, grapeseed oil, colza oil, cade oil, peach kernel oil, coffee bean oil, jojoba oil, and a mixture thereof.
1.34. Any of the preceding personal care compositions, wherein the personal care composition includes oil in an amount from about 0.1 to about 3 wt. %, based on the total weight of the personal care composition. In some embodiments, the total amount of oil(s) present in the personal care composition is from about 0.1 to about 3 wt. %, about 0.1 to about 2 wt. %, about 0.1 to about 1 wt. %, about 0.1 to about 0.5 wt. %; from about 0.4 to about 3 wt. %, about 0.4 to about 2 wt. %, about 0.4 to about 1 wt. %; from about 0.8 to about 3 wt. %, about 0.8 to about 2 wt. %; from about 1.4 to about 3 wt. %, about 1.4 to about 2 wt. %; from about 2 to about 3 wt. %, or any range or subrange thereof, based on the total weight of the personal care composition.
1.35. Any of the preceding personal care compositions, wherein the polyol(s) may be chosen from glycols or compounds with numerous hydroxyl groups. The term “polyol” should be understood as meaning, within the meaning of the present disclosure, an organic molecule comprising at least two free hydroxyl groups. The polyols of the personal care composition may be glycols or compounds with numerous hydroxyl groups. In some cases, the one or more polyols is/are selected from the group consisting of C₂-C₃₂ polyols. The one or more polyols may have from 2 to 32 carbon atoms, from 3 to 16 carbon atoms, or from 3 to 12 carbon atoms.
1.36. Any of the preceding personal care compositions, wherein the polyol(s) comprising ethylene glycol, propylene glycol, butylene glycol, hexylene glycol, glycerin, diglycerin, diethylene glycol, and dipropylene glycol, and mixtures thereof. In some cases, the polyol is butylene glycol. In some further cases, the personal care composition comprises at least butylene glycol, and optionally one or more polyols other than butylene glycol.
1.37. Any of the preceding personal care compositions, wherein the personal care composition comprises one or more polyol(s) in an amount from about 0.5 to about 10 wt. %, based on the total weight of the personal care composition. For example, the amount of polyol(s) present in the personal care composition may be from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %; from about 5 to about 10 wt. %, about 5 to about 8 wt. %; from about 7 to about 10%, about 8 to about 10 wt. %, or any range or subrange thereof, based on the total weight of the personal care composition.
1.38. Any of the preceding personal care compositions, wherein the composition comprises a humectant. Examples of humectants that may be incorporated in certain embodiments include triethylene glycol, tripropylene glycol, propylene glycol, glycerin, polyethylene glycols, polypropylene glycol, glycerin, sorbitol, hexylene glycol, butylene glycol, hyaluronic acid, 1,2 hexanediol, saccharide isomerate and a combination of two or more thereof. In some cases, the one or more humectants comprise at least one of glycerin, propylene glycol, or polyethylene glycol and, optionally, one or more additional humectants.
1.39. The preceding composition wherein the humectant is selected from glycerin, sorbitol, and a combination thereof (e.g., wherein the humectant is present from 1%-5% by wt.) (e.g., about 3% by wt.). For example, in some embodiments, the amount of humectant(s) in the personal care composition is from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; about 3 to about 5 wt. %, about 3 to about 4 wt. %, or any range or subrange thereof, based on the total weight of the personal care composition.
1.40. Any of the preceding personal care composition wherein composition comprises a pH modifying agent.
1.41. The preceding personal care composition, wherein the pH modifying agent is selected from: HCl, phosphoric acid, sulfonic acid, carboxylic acid (e.g., lactic acid and citric acid), acid salts (e.g., monosodium citrate, disodium citrate, sodium lactate, monosodium malate, etc.), alkali metal hydroxides (e.g., sodium hydroxide and potassium hydroxide), carbonates (e.g., sodium carbonate, bicarbonates, sesquicarbonates, borates, silicates, phosphates) (e.g., monosodium phosphate, trisodium phosphate, pyrophosphate salts, etc.), and imidazole.
1.42. The preceding personal care composition, wherein pH modifying agent is a carboxylic acid, and wherein the carboxylic acid comprises citric acid (e.g., from 0.1%-1% by wt.).
1.43. Any of the preceding personal care compositions, wherein the composition comprises water (e.g., from 10%-90% by wt.) (e.g., 25%-90%) (e.g., 50%-90%) (e.g., 50%-80%). For example, the amount of water present in the personal care composition may be from about 10 to about 90 wt. %, about 15 to about 90 wt. %, about 20 to about 90 wt. %, about 30 to about 90 wt. %, about 40 to about 90 wt. %, about 50 to about 90 wt. %, about 60 to about 90 wt. %, about 70 to about 90 wt. %, about 80 to about 90 wt. %; about 10 to about 80 wt. %, about 15 to about 80 wt. %, about 20 to about 80 wt. %, about 30 to about 80 wt. %, about 40 to about 80 wt. %, about 50 to about 80 wt. %, about 60 to about 80 wt. %, about 70 to about 80 wt. %; about 10 to about 70 wt. %, about 15 to about 70 wt. %, about 20 to about 70 wt. %, about 30 to about 70 wt. %, about 40 to about 70 wt. %, about 50 to about 70 wt. %, about 60 to about 70 wt. %; about 10 to about 60 wt. %, about 15 to about 60 wt. %, about 20 to about 60 wt. %, about 30 to about 60 wt. %, about 40 to about 60 wt. %, about 50 to about 60 wt. %; about 10 to about 50 wt. %, about 15 to about 50 wt. %, about 20 to about 50 wt. %, about 30 to about 50 wt. %, about 40 to about 50 wt. %, including any ranges and subranges thereof, based on the total weight of the personal care composition.
1.44. Any of the preceding personal care compositions, wherein the polyol is selected from: (e.g., glycerine, 1,3-propanediol, isoprene glycol, pentylene glycol, hexylene glycol, glycols such as ethylene glycol, propylene glycol, butylene glycol, diethylene glycol and dipropylene glycol, polyglycerols with 2 to 6 repeating units, for instance diglycerol, erythritol, arabitol, adonitol, sorbitol, dulcitol, glucose, fructose, xylose, trehalose, sucrose, maltose, saccharose and lactose, and mixtures thereof.).
1.45. The preceding personal care composition, wherein the polyol is butylene glycol (e.g., from 1 to 5 wt. % based on the weight of the personal care composition) (e.g., about 2 wt. % based on the weight of the personal care composition).
1.46. Any of the preceding personal care compositions, wherein the composition comprises a polymeric thickener.
1.47. The preceding composition, wherein the polymeric thickener comprises polyacrylate crosspolymer-6 (e.g., from 1 to 5 wt. % based on the weight of the personal care composition) (e.g., about 2 wt. % based on the weight of the personal care composition).
1.48. Any of the preceding compositions comprising an ingredient, such as a humectant, for moisturizing and/or promoting the moisturization of a keratin substrate (e.g., sometimes referred to as a “moisturizing ingredient”) selected from: glycerin, glyceryl oleate, caprylyl glycol, triglycerides (e.g., caprylic/capric triglyceride), silicone oils (e.g., cyclomethicone), ester oils (e.g., butyl myristate, isopropyl myristate, cetyl myristate, isopropyl palmitate, isopropyl stearate, octyl stearate, isocearyl stearate), organic fatty alcohols (e.g., oleic alcohol, linolenic alcohol, linoleic alcohol, isostearyl alcohol, octyl dodecanol), long chain fatty acids, liquid water-soluble polyols, propylene glycol, sorbitol, polyethylene glycol, ethoxylated/propoxylated ethers of methyl glucose (e.g., methyl gluceth-20) and ethoxylated/-propoxylated ethers of lanolin alcohol (e.g., Solulan-75), coco and tallow fatty acids, nonoclusive liquid water soluble polyols, essential amino acids, and sodium pyrrolidone carboxylic acid (or sodium dl-pyrrolidone carboxylate), urea, L-proline, guanidine and pyrrolidone; hexadecyl, myristyl, isodecyl or isopropyl esters of adipic, lactic, oleic, stearic, isostearic, myristic or linoleic acids and corresponding alcohol esters (e.g., sodium isostearoyl-2 lactylate, sodium capryl lactylate), aloe vera gel, acetamide MEA and combinations thereof. The foregoing ingredients for moisturizing and/or promoting the moisturization of a keratin substrate may be present in the personal care composition in any of the amounts disclosed for humectants, oils, and/or polyols depending on if such ingredient is a humectant, oil, and/or polyol.
1.49. The preceding composition wherein the moisturizing ingredient comprises caprylyl glycol (e.g., from 0.1 to 1 wt. % based on the weight of the personal care composition).
1.50. Any of the preceding personal care compositions comprising a preservative selected from: benzalkonium chloride; sodium benzoate, sodium salicylate; benzethonium chloride, 5-bromo-5-nitro-1,3dioxane; 2-bromo-2-nitropropane-1,3-diol; alkyl trimethyl ammonium bromide; N-(hydroxymethyl)-N-(1,3-dihydroxy methyl-2,5-dioxo-4-imidaxolidinyl-N-(hydroxy methyl)urea; 1-3-dimethyol-5,5-dimethyl hydantoin; formaldehyde; iodopropynyl butyl carbamate, butyl paraben; ethyl paraben; methyl paraben; propyl paraben, mixture of methyl isothiazolinone/methyl-chloroisothiazoline in a 1:3 wt. ratio; mixture of phenoxyethanol/butyl paraben/methyl paraben/propylparaben; 2-phenoxyethanol; tris-hydroxyethyl-hexahydrotriazine; methylisothiazolinone; 5-chloro-2-methyl-4-isothiazolin-3-one; 1,2-dibromo-2,4-dicyanobutane; 1-(3-chloroalkyl)-3,5,7-triaza-azoniaadamantane chloride; organic acids, lactic acid, or citric acid and combinations thereof.
1.51. The preceding composition, wherein the preservative comprises sodium benzoate.
1.52. Any of the preceding personal care compositions, wherein the composition comprises one or more amino acids selected from: arginine, lysine, serine, citrulline, ornithine, creatine, histidine, diaminobutanoic acid, diaminoproprionic acid, alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, salts thereof, and combinations thereof.
1.53. The preceding composition, wherein the composition comprises taurine, arginine, and glycine.
1.54. The preceding personal care composition, wherein the taurine: arginine: glycine are in a weight ratio of (1.5-69):(0.5-40):(0.5-1.5), respectively. For example, the personal care composition may be formulated to have a weight ratio of taurine: arginine: glycine may be from about 100:50:1 to about 30:15:1; about 80:45:1 to about 30:15:1, about 70:40:1 to about 30:15:1, about 65:34:1 to about 30:15:1, about 55:25:1 to about 30:15:1; from about 100:50:1 to about 40:20:1; about 80:45:1 to about 40:20:1, about 70:40:1 to about 40:20:1, about 65:34:1 to about 40:20:1, about 55:25:1 to about 40:20:1; from about 100:50:1 to about 50:30:1; about 80:45:1 to about 50:30:1, about 70:40:1 to about 50:30:1, about 65:34:1 to about 50:30:1, about 55:25:1 to about 50:30:1; from about 100:50:1 to about 60:35:1; about 80:45:1 to about 60:35:1, about 70:40:1 to about 60:35:1, about 65:34:1 to about 60:35:1, about 55:25:1 to about 60:35:1, or any range or subrange thereof, based on the total weight of the personal care composition.
1.55. The preceding composition, wherein the taurine: arginine: glycine are in a weight ratio of about (65):(34):(1), respectively.
1.56. Any of the preceding compositions, wherein the composition comprises:
   an effective amount of butylene glycol (e.g., from 1 to 5 wt. % based on the weight of the personal care composition) (e.g., about 2 wt. % based on the weight of the personal care composition); and
   Wherein the surfactant system comprises:
   an amphoteric surfactant, wherein the amphoteric surfactant comprises cocamidopropyl hydroxysultaine,
   an alkyl glucoside, wherein the alkyl glucoside comprises a combination of decyl glucoside and caprylyl/capryl glucoside ((e.g., wherein the total combination is from 1 to 10 wt. % based on the weight of the personal care composition) (e.g., about 8 wt. % based on the weight of the personal care composition)),
   a non-ionic surfactant, wherein the non-ionic surfactant is a blend of decyl glucoside and sodium lauroyl lactylate (e.g., PUREACT 138) (e.g., wherein the surfactant is present from e.g., wherein the surfactant is present from 1 to 5 wt. % based on the weight of the personal care composition) (e.g., about 2 wt. % based on the weight of the personal care composition),
   an anionic surfactant, wherein the anionic surfactant comprises sodium cocoyl apple amino acids (e.g., from 1 to 10 wt. % based on the weight of the personal are composition) (e.g., about 5 wt. % based on the weight of the personal care composition);
glycerin (e.g., from 1 to 5 wt. % based on the weight of the personal care composition) (e.g., about 3 wt. % based on the weight of the personal care composition);
polyacrylate crosspolymer-6 (e.g., from 1 to 5 wt. % based on the weight of the personal are composition) (e.g., about 2 wt. % based on the total weight of the personal care composition); and
water (e.g., from 50 to 75 wt. % based on the weight of the personal care composition) (e.g., about 70 wt. % based on the total weight of the personal care composition).
1.57. Any of the preceding personal care compositions, wherein the composition is free or substantially free of sodium lauryl sulfate (SLS). In some embodiments, the personal care composition is formulated to have an amount of sodium lauryl sulfate and/or sodium laureth sulfate of about 4 wt. % or less, about 3 wt. % or less, about 2 wt. % or less, about 1 wt. % or less, about 0.5 wt. % or less, about 0.1 wt. % or less, based on the total weight of the personal care composition.
1.58. Any of the preceding personal care compositions, wherein the personal care composition is a rinse off composition, optionally wherein the composition is a liquid soap, liquid hand soap, a facial cleanser and/or facial soap, shower gel, body wash, and shampoo.
1.59. Any of the preceding personal care compositions, wherein the composition is a foaming facial cleanser.
1.60. Any of the preceding personal care compositions, wherein the personal care composition comprises an additional active ingredient selected from: carnosine, cica (asiaticoside), hyaluronic acid and salts thereof (e.g., sodium hyaluronate), saccharide isomerate, bromelain enzyme, pumpkin enzyme, zingiber officinale (ginger) root extract, bisabolol, asiaticoside, tocopherol, vitamin E acetate, vitamin C, and combinations thereof. The additional active ingredients may be present in the personal care compositions in an amount from about 0.01 to about 5 wt. %, based on the total weight of the personal care composition. For instance, amount of additional ingredients present in the personal care composition is from about 0.01 to about 4 wt. %, about 0.01 to about 3 wt. %, about 0.01 to about 2 wt. %, about 0.01 to about 1 wt. %, about 0.01 to about 0.5 wt. %, about 0.01 to about 0.1 wt. %; about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.5 wt. %, about 0.1 to about 0.1 wt. %; about 0.5 to about 5 wt. %, about 0.5 to about 4 wt. %, about 0.5 to about 3 wt. %, about 0.5 to about 2 wt. %, about 0.5 to about 1 wt. %; about 0.75 to about 5 wt. %, about 0.75 to about 4 wt. %, about 0.75 to about 3 wt. %, about 0.75 to about 2 wt. %, about 0.75 to about 1 wt. %; about 1 to about 5 wt. %, about 1 to about 4 wt. %, about 1 to about 3 wt. %, about 1 to about 2 wt. %; about 2 to about 5 wt. %, about 2 to about 4 wt. %, about 2 to about 3 wt. %; about 3 to about 5 wt. %, about 3 to about 4 wt. %, including any range or subrange therebetween, based on the total weight of the personal care composition.
1.61. Any of the preceding personal care compositions, wherein the personal care compositions preferably produces a foam having an elastic modulus of about 10 to about 50 Pa, e.g. from about 20 to 45 or about 25 to 45, at a stress of at least 8 Pa. Additionally or alternatively, the personal care compositions may produce a foam having an elasticity of about 25 to 50 Pa, preferably about 30 to about 50 Pa, about 30 to about 45 Pa, or about 32 to about 45 Pa, e.g., as assessed by an ANOVA test. In some embodiments, the personal care compositions may produce a foam having a yield stress of about 4 to about 10 Pa, e.g., from about 5 to about 9 Pa, or about 5.5 to about 8 Pa, e.g., as assessed by an ANOVA test.

The present invention provides, in another aspect, a personal care composition (Composition 2.0), e.g., any of Composition 1.0 et seq, for use in topical application to the skin.

In some aspects, the personal care compositions of the disclosure, e.g., any of Composition 1.0 et seq., can comprise an oil selected from: sunflower seed oil, meadowfoam oil, olive oil, shear butter, jojoba oil, almond oil, grape seed oil, rose hip seed oil, mink oil, castor oil, soybean oil, mineral oil, and combinations thereof.

In some embodiments, the personal care compositions of the disclosure, e.g., any of Composition 1.0 et seq., are free or substantially free of sodium lauryl sulfate (SLS). In some embodiments, the composition (e.g., any of Composition 1.0 et seq) is free or substantially free of sodium lauryl ether sulfate (SLES). In some embodiments, the composition is free or substantially free of sodium lauryl sulfate (SLS) and sodium lauryl ether sulfate (SLES). In some embodiments, the composition is free or substantially free of alkyl sulfate salts, e.g., C₁₋₂₅ alkyl sulfate salts. In some embodiments, the composition is free or substantially free of alkyl aryl sulfonate salts, e.g., C₁₋₂₅ alkyl aryl sulfonate salts, e.g., alkyl benzene sulfonate salts, e.g., sodium dodecyl benzene sulfonate. In some embodiments, the composition is free or substantially free of alkyl sulfate salts, e.g., C₁₋₂₅ alkyl sulfate salts, and alkyl aryl sulfonate salts, e.g., C₁₋₂₅ alkyl aryl sulfonate salts.

In some embodiments, e.g., any of Composition 1.0 et seq, the composition is free or substantially free of a sulfate based surfactant. Additionally or alternatively, in some embodiments, e.g., any of Composition 1.0 et seq, the composition is free or substantially free of sulfate.

The composition of the present disclosure (e.g., any of Composition 1.0 et seq) may be any type of personal care composition. In certain embodiments, the composition is formulated to be a topical skin care formulations suitable for application to skin. Examples of such compositions include, but are not limited to, personal care compositions, skin care compositions (e.g., foaming facial cleansers), liquid hand soap, body washes, creams, shower gels, bar soaps, shampoo, and cosmetics. In some embodiments, the composition is a rinse off product (liquid soap, liquid hand soap, shower gel, body wash, shampoo, etc.). The composition (e.g., any of Composition 1.0 et seq) can comprise a single phase or can be a multi-phase system, for example a system comprising a polar phase and an oil phase, optionally in the form of a stable emulsion. For example, the personal care composition may be in the form of an oil-in-water emulsion.

The personal care composition (e.g., any of Composition 1.0 et seq) can be liquid, semi-solid or solid. The formulation can be provided in any suitable container such as an aerosol can, tube or container with a porous cap, roll-on container, bottle, container with an open end, etc.

The personal care composition may be adapted to be dispensed from a pump in the form of a foam. It was unexpected discovered that certain embodiments of the personal care composition can be dispensed as a foam having preferable characteristics using a mechanical pump, such as a mechanical foamer pump. The mechanical pump may be a mechanical foamer pump having a foaming chamber, which may contain a mesh therein. The mechanical foaming chamber may have an inlet and a foam outlet, wherein the cross-sectional area of the foam outlet is greater than the cross-sectional area of the inlet by about 100 to about 500%. In some instances, the cross-sectional area of the foam outlet is greater than the cross-sectional area of the inlet by about 100 to about 500%, about 125 to about 500%, about 150 to about 500%, about 175 to about 500%, about 200 to about 500%, about 225 to about 500%, about 250 to about 500%, about 275 to about 500%, about 300 to about 500%, about 325 to about 500%, about 350 to about 500%, about 375 to about 500%, about 400 to about 500%, about 425 to about 500%; about 100 to about 400%, about 125 to about 400%, about 150 to about 400%, about 175 to about 400%, about 200 to about 400%, about 225 to about 400%, about 250 to about 400%, about 275 to about 400%, about 300 to about 400%, about 325 to about 400%; about 100 to about 300%, about 125 to about 300%, about 150 to about 300%, about 175 to about 300%, about 200 to about 300%, about 225 to about 300%, or any range or subrange thereof.

The mesh contained in the foaming chamber may be selected to promote foaming of personal care composition. It was determined that certain meshes are configured to promote mixing of the certain personal care composition disclosed herein with air to promote the production of foam having certain desirable characteristics. The mesh may be configured as a mesh screen, a mesh ball, or the like.

Without being limited to any specific theory, it is believed that a personal care product comprising a container comprising certain mechanical pumps in conjunction with certain personal care compositions can produce a foam from the personal care composition having certain desirable foam characteristics including, e.g., a preferable elastic modulus, preferable elasticity, and/or preferable yield stress. For instance, the personal care composition when dispensed with the mechanical pump may produce a foam having an elastic modulus of about 10 to about 50 Pa, e.g. from about 20 to 45 or about 25 to 45, at a stress of at least 8 Pa. Additionally or alternatively, the personal care composition when dispensed with the mechanical pump may produce a foam having an elasticity of about 25 to 50 Pa, preferably about 30 to about 50 Pa, about 30 to about 45 Pa, or about 32 to about 45 Pa, e.g., as assessed by an ANOVA test. In some embodiments, the personal care composition when dispensed with the mechanical pump may produce a foam having a yield stress of about 4 to about 10 Pa, e.g., from about 5 to about 9 Pa, or about 5.5 to about 8 Pa, e.g., as assessed by an ANOVA test. Although certain embodiments of the personal care composition are adapted for dispensing as a foam, in other embodiments, the personal care composition may be dispensed in the form of a liquid or a gel.

Water may be present in the compositions of the disclosure (e.g., any of Composition 1.0 et seq). Water employed in the preparation of commercial personal care compositions should be deionized and free of organic impurities. Water can make up the balance of the compositions and be present in an amount from 10 to 90 wt. %, or 10 to 80 wt. %, based on the weight of the personal care compositions. The total amount of water includes the water which is added plus that amount which is introduced with other materials such as glycerin, sorbitol or any components of the personal care composition.

In some embodiments the composition of the disclosure (e.g., any of Composition 1.0 et seq) provides a sulfate-free personal care formulation comprising a surfactant system, humectants, antioxidants, pH adjusters, vitamins, fragrances, thickeners, oils, and water.

In some aspects, the compositions of the disclosure, e.g., any of Composition 1.0 et seq., comprise a pH adjuster or modifying agent (also referred to herein as pH modifier). The pH adjusters or pH modifying agents of the present disclosure can comprise but are not limited to lactic acid, sodium hydroxide, citric acid and combinations thereof. In one aspect, pH modifying agents include acidifying agents to lower pH, basifying agents to raise pH and buffering agents to control pH within a desired range. For example, one or more compounds selected from acidifying, basifying and buffering agents can be included to provide a pH of 2 to 10, or in various illustrative embodiments 2 to 8, 3 to 9, 4 to 8, 5 to 7, 6 to 10, 7 to 9, etc. In some preferred embodiments, the pH is between about 1 to 5, about 2 to 5, about 4 to 5, or about 4.2-4.8. Examples of pH modifying agent include HCl, phosphoric and sulfonic acids and carboxylic acids such as lactic acid and citric acid, acid salts (e.g., monosodium citrate, disodium citrate, sodium lactate, monosodium malate, etc.), alkali metal hydroxides such as sodium hydroxide and potassium hydroxide, carbonates such as sodium carbonate, bicarbonates, sesquicarbonates, borates, silicates, phosphates (e.g., monosodium phosphate, trisodium phosphate, pyrophosphate salts, etc.), imidazole and combinations thereof. One or more pH modifying agents are optionally present in a total amount effective to maintain the composition in an acceptable pH range. In some aspects, only one or two pH modifying agents are employed. In some preferred embodiments of the personal care composition, which may be in the form of a shampoo, shower gel and face wash compositions, the pH modifier is lactic acid, sodium lactate, or a combination of both lactic acid and sodium lactate.

In certain aspects the compositions of the present disclosure, e.g., any of Composition 1.0 et seq, provide for a sulfate-free personal care formulation comprising an antibacterial agent. The antibacterial agent of the invention may be lactic acid.

In some aspects, the personal care compositions of the present disclosure, e.g., any of Composition 1.0 et seq, contain a long chain alkyl anionic surfactant. In at least one aspect, the long chain alkyl anionic surfactant is a C₆-C₂₂ anionic surfactant that comprises a surfactant selected from the group consisting of: long chain alkyl sulfonates, long chain alkyl phosphates, long chain alkyl alpha olefin sulfonates, long chain alkyl taurates, long chain alkyl isethionates (SCI), long chain alkyl glyceryl ether sulfonates (AGES), sulfosuccinates, sodium methyl-2 sulfolaurate, disodium 2-sulfolaurate (e.g., TEXAPON® SFA produced by BASF), sodium lauryl sulfoacetate blend, and combinations thereof. Examples of acyl taurates include those of the following formula:

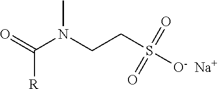

wherein R, R¹, R² and R³ are each independently selected from H or an alkyl chain having 1-24 carbon atoms, or from 6-20 carbon atoms, or from 8 to 16 carbon atoms, said chain being saturated or unsaturated, linear or branched, and X is COO⁻ or SO₃⁻. Non-limiting examples of acyl taurate salts include sodium cocoyl taurate, sodium methyl cocoyl taurate, oleoyl taurate, and combinations thereof. In at least one preferred embodiment, the personal care composition includes one or more anionic surfactant comprising sodium n-coconut acid-n-methyl taurate.

In some aspects, the compositions of the present disclosure, e.g., any of Composition 1.0 et seq, comprise one or more anionic surfactants that are anionic derivatives of proteins of plant origin. In some aspects, the anionic derivatives of proteins of plant origin can be protein hydrolysates containing a hydrophobic group. In some aspects the hydrophobic group may be naturally present in the protein and in other aspects it may be added by reacting the protein and/or the protein hydrolysate with a hydrophobic compound. In certain aspects the proteins are of plant origin, or are derived from silk, and the hydrophobic group be a fatty chain (e.g., an alkyl chain comprising from 10 to 22 carbon atoms). In yet a further aspect, the compositions of the disclosure (e.g., any of Composition 1.0 et seq) comprises an anionic derivative of proteins of plant origin from apple, wheat, soybean or oat protein hydrolysates comprising an alkyl chain having from 10 to 22 carbon atoms and salts thereof. In some aspects the alkyl chain may especially be a lauryl chain and the salt may be a sodium, potassium and/or ammonium salt.

In yet a further aspect, the compositions of the disclosure (e.g., any of Composition 1.0 et seq) comprise an anionic derivative of proteins of plant or silk origin wherein the protein is a salt of protein hydrolysates where: the protein is a silk protein modified by lauric acid; salts of protein hydrolysates where the protein is a wheat protein modified by lauric acid, (e.g., potassium lauroyl wheat amino acids) or the sodium salt (e.g., sodium lauroyl wheat amino acids); salts of protein hydrolysates where the protein is an oat protein and where the protein is an oat protein modified by lauric acid (e.g., a sodium salt thereof, such as sodium lauroyl oat amino acids); or salts of apple protein hydrolysates (e.g., a sodium salt thereof, such as sodium cocoyl apple amino acids). In some aspects, the anionic derivative can comprise a mixture of lauroyl amino acids (aspartic acid, glutamic acid, glycine, alanine) neutralized with sodium N-methylglycinate (e.g., sodium cocoyl amino acids and/or sodium cocoyl glycinate).

The compositions of the disclosure, e.g., any of Composition 1.0 et seq, comprise one or more alkyl glucoside(s). An alkyl glucoside is a compound produced by combining a sugar such as glucose with a fatty alcohol. Alkyl refers to unbranched or branched carbon chain. In some embodiments, the alkyl group is unbranched. The alkyl group may be saturated or unsaturated. In some embodiments, the alkyl group is saturated. For example, the structure of decyl glucoside is shown below:

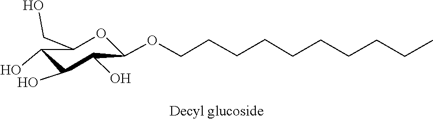

The one or more alkyl glucoside(s) may be comprise a C₈₋₂₅ alkyl glucoside, e.g., a C₈₋₁₈ alkyl glucoside, a C₁₀₋₁₈ alkyl glucoside, a C₁₀₋₁₆ alkyl glucoside, or a combination of two or more thereof. In some embodiments, the alkyl glucoside is selected from decyl glucoside, caprylyl/capryl glucoside, lauryl glucoside, coco-glucoside, octyl glucoside, cetearyl glucoside, cetyl glucoside, hexadecyl glucoside, arachidyl glucoside, and a combination thereof. In some embodiments, the alkyl glucoside is selected from decyl glucoside, caprylyl/capryl glucoside and a combination thereof. In some embodiments, the weight ratio of caprylyl/capryl glucoside to decyl glucoside present in the composition is 2:1 to 1:1 or 4:1 to 1:1, e.g., about 3:1 to 1:1. In some embodiments, the alkyl glucoside is present in an amount of from 1 to 10 wt. %, e.g., from 2 to 9 wt. %, from 2 to 7 wt. %, based on the total weight of the personal care composition.

In some embodiments, the personal care compositions of the disclosure (e.g., any of Composition 1.0 et seq) can comprise a C8-C16 aminopropyl betaine surfactant, e.g., cocamidopropyl betaine. In some embodiments, the personal compositions of the disclosure (e.g., any of Composition 1.0 et seq) can comprise a non-ionic block copolymer, optionally together with an alkyl glucoside. The non-ionic block copolymer may be a poly(propylene oxide)/poly(ethylene oxide) copolymer. In some embodiments, the copolymer has a polyoxypropylene molecular mass of from 3000 to 5000 g/mol and a polyoxyethylene content of from 60 to 80 mol. %. In some embodiments, the non-ionic block copolymer is a poloxamer. In some embodiments, the non-ionic block copolymer is selected from: Poloxamer 338, Poloxamer 407, Poloxamer, 237, Poloxamer, 217, Poloxamer 124, Poloxamer 184, Poloxamer 185, and a combination of two or more thereof. In some embodiments, the copolymer is Poloxamer 407. In some embodiments, the compositions of the disclosure (e.g., any of Composition 1.0 et seq) can comprise a betaine amphoteric surfactant and a non-ionic block copolymer, optionally together with an alkyl glucoside.

In some embodiments, the personal care composition of the disclosure (e.g., any of Composition 1.0 et seq) may be free or substantially free of sodium lauryl sulfate (SLS) and/or sodium lauryl ether sulfate (SLES). In some embodiments, the composition may be free or substantially free of alkyl sulfate salts. In some embodiment, the alkyl sulfate salts are C₁₋₂₅ alkyl sulfate which may be saturated or unsaturated, and unbranched or branched. In some embodiments, the personal care composition may be free or substantially free of alkyl aryl sulfonate salts, e.g., alkyl benzene sulfonate salts, e.g., sodium dodecyl benzene sulfonate. In some embodiments, the personal care composition may be free or substantially free of alkyl sulfate salts and alkyl aryl sulfonate salts. In some embodiments, the composition may be free or substantially free of sulfate. As used herein, “substantially free” of a material may refer to a composition where the material is present in an amount of less than 0.1 weight %, less than 0.05 weight %, less than 0.01 weight %, less than 0.005 weight %, less than 0.001 weight %, or less than 0.0001 weight % based on a total weight of the composition.

In some aspects, the personal care compositions of the present disclosure, (e.g., any of Composition 1.0 et seq), comprise one or more topically active compounds. Topically active compounds may encompass a wide range of materials, including antibacterial agents, vitamins, medicaments, fragrance materials, antioxidants and other skin-care ingredients. In some aspects, the personal care composition of the present disclosure, e.g., any of Composition 1.0 et seq, comprises a vitamin. Illustrative vitamins may be or include, but are not limited to, vitamin C, vitamin D, vitamin E, vitamin K, and a combination thereof.

In certain aspects, optional ingredients of the personal care compositions of the disclosure, e.g., any of Composition 1.0 et seq, include solvents; water-soluble alcohols such as C₂₋₈ alcohols including ethanol; glycols including propylene glycol, dipropylene glycol, tripropylene glycol and mixtures thereof; glycerides including mono-, di-and triglycerides; medium to long chain organic acids, alcohols and esters; surfactants including emulsifying and dispersing agents; amino acids including glycine; emollients; fragrances; and colorants including dyes and pigments.

In certain aspects, the compositions of the disclosure, e.g., any of Composition 1.0 et seq, may optionally contain emollients in any desired amount to achieve a desired emollient effect. Emollients are known in the art and are used to impart a soothing effect on the skin. In some aspects non-volatile emollients can be used. Non-volatile, non-silicone emollients include C₁₂₋₁₅ alkyl benzoate. The non-volatile silicone material can be a polyethersiloxane, polyalkyarylsiloxane or polyethersiloxane copolymer. An illustrative non-volatile silicone material is phenyl trimethicone. Examples of emollients include, but are not limited to, PPG-14 butyl ether, PPG-3 myristyl ether, secondary alcohol ethoxylates, stearyl alcohol, stearic acid and salts thereof, glyceryl monoricinoleate, isobutyl palmitate, glyceryl monostearate, isocetyl stearate, sulphated tallow, oleyl alcohol, propylene glycol, isopropyl laurate, mink oil, sorbitan stearate, cetyl alcohol, hydrogenated castor oil, stearyl stearate, hydrogenated soy glycerides, isopropyl isostearate, hexyl laurate, dimethyl brassylate, decyl oleate, diisopropyl adipate, n-dibutyl sebacate, diisopropyl sebacate, 2-ethyl hexyl palmitate, isononyl isononanoate, isodecyl isononanoate, isotridecyl isononanoate, 2-ethyl hexyl palmitate, 2-ethyl hexyl stearate, Di-(2-ethyl hexyl) adipate), Di-(2-ethyl hexyl) succinate, isopropyl myristate, isopropyl palmitate, isopropyl stearate, octacosanol, butyl stearate, glyceryl monostearate, polyethylene glycols, oleic acid, triethylene glycol, lanolin, castor oil, sunflower seed oil, acetylated lanolin alcohols, acetylated lanolin, petrolatum, isopropyl ester of lanolin, fatty acids, mineral oils, butyl myristate, isostearic acid, palmitic acid, PEG-23 oleyl ether, olelyl oleate, isopropyl linoleate, cetyl lactate, lauryl lactate, myristyl lactate, quaternised hydroxy alkyl, aminogluconate, vegetable oils, isodecyl oleate, isostearyl neopentanoate, myristyl myristate, oleyl ethoxy myristate, diglycol stearate, ethylene glycol monostearate, myristyl stearate, isopropyl lanolate, paraffin waxes, glycyrrhizic acid, hydrocyethyl stearate amide.

In some embodiments, the personal care composition of the disclosure, e.g., any of Composition 1.0 et seq, comprises an oil selected from sunflower seed oil, olive oil, shear butter, jojoba oil, almond oil, grape seed oil, rose hip seed oil, mink oil, castor oil, soybean oil, mineral oil, and a combination thereof. In certain embodiment, the personal care composition comprises sunflower seed oil.

In certain aspects, the personal care composition may include one or more humectants. Humectants can reduce evaporation and also contribute towards preservation by lowering water activity and can also impart desirable sweetness or flavor to compositions. Illustrative humectants may be or include, but are not limited to, glycerin, propylene glycol, polyethylene glycol, sorbitol, xylitol, or the like, or any mixture or combination thereof. In some embodiment, the humectant is selected from glycerin, sorbitol and a combination thereof. In certain embodiment, the humectant is glycerin.

Antioxidants may be added to the personal care composition, preferably to act as ingredient protectants and for maintenance of long-term stability of the composition. Examples of antioxidants include, but are not limited to citric acid, butylated hydroxytoluene, pentaerythrityl tetra-di-t-butyl hydroxyhydrocinnamate.

In certain aspects, the personal care compositions of the disclosure may optionally contain polymeric materials for thickening, such as natural or synthetic gums, such as polyglycerides including agar, agarose, pectin, or guars, polysaccharides, associative thickeners (e.g., PEG150 distearate) or combinations thereof. One class of materials worthy of attention for thickening a water-immiscible phase comprises derivatives of hydrolysed starch or other polysaccharides, including in particular esterified dextrins, such as dextrin palmitate. Suspending agents such as silicas or clays such as bentonite, montmorillonite or hectorite, including those available under the trademark Bentone can also be employed to thicken liquid compositions according to the invention. The personal care composition can be thickened with non-polymeric organic gellants, including selected dibenzylidene alditols (e.g., dibenzylidene sorbitol).

In certain aspects, the personal care compositions of the disclosure, e.g., any of Composition 1.0 et seq., may include a thickening system having one or more thickeners. The one or more thickeners may be configured to at least partially form a network and thereby prevent phase separation in the personal care composition. Illustrative thickeners may be or include a polymeric thickener, such as a copolymer of at least two monomers, where a first monomer may be an acrylamide having a sulfonic or sulphonic group, and a second monomer may be a vinyl monomer having a nitrogenous side chain. Illustrative vinyl monomers may be or include, but are not limited to, methacrylic acid, methacrylates, methacrylamides, methacrylate salts, vinyl pyrrolidone, and the like, and combinations thereof. For example, the thickener may be or include a copolymer of 2-acrylamidomethylpropanesulphonic acid or a salt thereof, such as an ammonium or sodium salt, and a vinyl monomer having a nitrogenous cyclic side chain or a vinyl monomer having an amide functional group, such as N-vinylpyrrolidone. In a preferred implementation, the thickener may be a copolymer of the ammonium salt of 2-acrylamidomethylpropanesulphonic acid and N-vinylpyrrolidone. The one or more thickener(s) may comprise a block copolymer thickener, such as ammonium acryloyldimethyltaurate/VP copolymer. In another embodiment, the thickener may be a block copolymer of the sodium salt of 2-aoylamidomethylpropanesulphonic acid and N-vinylpyrrolidone, such as sodium acryloyldimethyltaurate/VP crosspolymer. In yet another aspect, the compositions of the disclosure, e.g., any of Composition 1.0 et seq, comprise a polymeric thickener that is selected from ARISTOFLEX® HMB, ARISTOFLEX® BLV, and/or ARISTOFLEX® TAC, which are commercially available from Clariant International Ltd. In a further aspect, the compositions of the disclosure, e.g., any of Composition 1.0 et seq, comprise polyacrylate crosspolymer-6.

In some embodiments, the personal care compositions of the disclosure can be in the form of a shampoo, a shower gel, or a face wash composition. In these aspects, the personal care compositions of the present disclosure can optionally include an emollient or moisturizer component. Illustrative examples of such emollient or moisturizer components include glycerin, glyceryl oleate, caprylyl glycol, triglycerides (e.g., caprylic/capric triglyceride), silicone oils (e.g., cyclomethicone), ester oils (e.g., butyl myristate, isopropyl myristate, cetyl myristate, isopropyl palmitate, isopropyl stearate, octyl stearate, isocearyl stearate), organic fatty alcohols (e.g., oleic alcohol, linolenic alcohol, linoleic alcohol, isostearyl alcohol, octyl dodecanol), long chain fatty acids, liquid water-soluble polyols, propylene glycol, sorbitol, polyethylene glycol, ethoxylated/propoxylated ethers of methyl glucose (e.g., methyl gluceth-20) and ethoxylated/-propoxylated ethers of lanolin alcohol (e.g., Solulan-75), coco and tallow fatty acids, 31 onocclusive liquid water soluble polyols, essential amino acids, and sodium pyrrolidone carboxylic acid (or sodium dl-pyrrolidone carboxylate), urea, L-proline, guanidine and pyrrolidone; hexadecyl, myristyl, isodecyl or isopropyl esters of adipic, lactic, oleic, stearic, isostearic, myristic or linoleic acids and corresponding alcohol esters such as sodium isostearoyl-2 lactylate, sodium capryl lactylate, aloe vera gel and acetamide MEA.

In some embodiments, preservatives are included in the present personal care compositions (e.g., any of Composition 1.0 et seq) can be from a concentration of about 0.01 wt. % to about 10 wt. %, about 0.01 wt. % to 3 wt. %, or 0.01 wt. % to 2.5 wt. %, based on the total weight of the personal care composition. In some aspects, the personal care compositions of the disclosure, e.g., any of Composition 1.0 et seq, can be selected from: benzalkonium chloride; sodium benzoate, sodium salicylate; benzethonium chloride, 5-bromo-5-nitro-1,3dioxane; 2-bromo-2-nitropropane-1,3-diol; alkyl trimethyl ammonium bromide; N-(hydroxymethyl)-N-(1,3-dihydroxy methyl-2,5-dioxo-4-imidaxolidinyl-N-(hydroxy methyl)urea; 1-3-dimethyol-5,5-dimethyl hydantoin; formaldehyde; iodopropynyl butyl carbamate, butyl paraben; ethyl paraben; methyl paraben; propyl paraben, mixture of methyl isothiazolinone/methyl-chloroisothiazoline in a 1:3 wt. ratio; mixture of phenoxyethanol/butyl paraben/methyl paraben/propylparaben; 2-phenoxyethanol; tris-hydroxyethyl-hexahydrotriazine; methylisothiazolinone; 5-chloro-2-methyl-4-isothiazolin-3-one; 1,2-dibromo-2,4-dicyanobutane; 1-(3-chloroalkyl)-3,5,7-triaza-azoniaadamantane chloride; organic acids, lactic acid, citric acid and combinations thereof.

In some aspects, the personal care composition of the disclosure, e.g., any of Compositions 1.0 et seq, can comprise a basic amino acid. The basic amino acids which can be used in the personal care compositions and methods of the invention include not only naturally occurring basic amino acids, such as arginine, lysine, and histidine, but also any basic amino acids having a carboxyl group and an amino group in the molecule, which are water-soluble and provide an aqueous solution with a pH of 7 or greater.

For example, basic amino acids include, but are not limited to, arginine, lysine, serine, citrulline, ornithine, creatine, histidine, diaminobutanoic acid, diaminoproprionic acid, salts thereof or combinations thereof. In a particular embodiment, the basic amino acids are selected from arginine, citrulline, and ornithine. In certain embodiments, the basic amino acid is arginine, for example, L-arginine, or a salt thereof.

In another aspect, the personal care compositions of the disclosure (e.g., Compositions 1.0 et seq) can further comprise one or more neutral amino acid, which can include, but is not limited to, one or more neutral amino acids selected from the group consisting of alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, and combinations thereof.

In some aspects, fragrance may be included in the personal care composition of the disclosure, e.g., any of Composition 1.0 et seq. Any fragrance suitable for personal care use may be incorporated into the personal care composition of the invention. Fragrances tend to be relatively volatile aroma compounds which are capable of entering the gas phase at skin surface temperature.

The personal care compositions of the disclosure may be manufactured using methods known in the art. Typically, the ingredients are combined and optionally heated where components need to be melted. The components are mixed. Desirably, volatile materials such as fragrant materials are incorporated in the composition in the latter stages of a mixing cycle in order to avoid volatilization thereof. After mixing, the composition may be poured directly into the dispensers and the container capped to preserve the product until use.

In another aspect, the invention provides a method of depositing a topically active compound on the skin, comprising applying an effective amount of any of personal care compositions disclosed herein, e.g., any of Compositions 1 et seq., to the skin.

Non-limiting embodiments of the personal care composition are described below.

In a first embodiment, provided is a personal care composition comprising:
a polyol;
a surfactant system, wherein the surfactant system comprises:
   one or more alkyl glucoside(s),
   an amphoteric surfactant,
   an anionic surfactant selected from an anionic derivatives of proteins of plant origin, anionic derivatives of proteins of silk protein, a combination of two or more thereof, and
   a non-ionic surfactant; and
water.

According to a second embodiment, the personal care composition of the first embodiment, wherein the amphoteric surfactant is a sultaine surfactant.

According to a third embodiment, the personal care composition of the second embodiment, wherein the sultaine surfactant is selected from the group consisting of lauramidopropyl hydroxysultaine, cocamidopropyl hydroxysultaine, oleamidopropyl hydroxysultaine, tallowamidopropyl hydroxysultaine, erucamidopropyl hydroxysultaine, lauryl hydroxysultaine, lauramidopropyl hydroxysultaine, and a combination of two or more thereof.

According to a fourth embodiment, the personal care composition of the third embodiment, wherein the sultaine surfactant comprises cocamidopropyl hydroxysultaine.

According to a fifth embodiment, the personal care composition of any preceding embodiment, wherein the alkyl glucoside is C₆₋₂₅ alkyl glucoside.

According to a sixth embodiment, the personal care composition of any preceding embodiment, wherein the alkyl glucoside is selected from decyl glucoside, caprylyl/capryl glucoside, lauryl glucoside, coco-glucoside, octyl glucoside, cetearyl glucoside, cetyl glucoside, hexadecyl glucoside, arachidyl glucoside, and a combination of two more thereof.

According to a seventh embodiment 7, the personal care composition of any preceding embodiment, wherein the alkyl glucoside comprises a combination of decyl glucoside and caprylyl/capryl glucoside.

According to an eight embodiment, the personal care composition of any preceding embodiment, wherein the anionic surfactant is an anionic derivative of a protein hydrolysate of plant origin, and wherein the protein hydrolysate comprises a hydrophobic group.

According to a ninth embodiment, the personal care composition of the eight embodiment, wherein the protein hydrolysate is an apple, a wheat, a soybean, or an oat protein hydrolysate comprising an alkyl chain having from 10 to 22 carbon atoms, and salts thereof.

According to a tenth embodiment, the personal care composition of the ninth embodiment, wherein the protein hydrolysate is an apple protein hydrolysate comprising an alkyl chain having from 10 to 22 carbon atoms.

According to an eleventh embodiment, the personal care composition of the tenth embodiment, wherein the anionic surfactant comprises sodium cocoyl apple amino acids.

According to a twelfth embodiment, the personal care composition of any preceding embodiment, wherein the non-ionic surfactant comprises a blend of an alkyl glucoside and a lactylate having carbon chains between C₈ and C₁₆ and wherein the lactylate is selected from the group consisting of ammonium, sodium, potassium and alkanolamine (e.g., triethanolamine) salts of lauroyl lactylate, cocoyl lactylate, lauroyl lactylate, caproyl lactylate, and a combination of two or more thereof.

According to a thirteenth embodiment, the personal care composition of the twelfth embodiment, wherein alkyl glucoside comprises decyl glucoside.

According to a fourteenth embodiment, the personal care composition of the twelfth or thirteenth embodiment, wherein the lactylate comprises sodium lauroyl lactylate.

According to a fifteenth embodiment, the personal care composition of one of the twelfth to the fourteenth embodiment, wherein the non-ionic surfactant is a blend of an alkyl glucoside and a lactylate having carbon chains between C₈ and C₁₆, and wherein the surfactant is a blend of decyl glucoside and sodium lauroyl lactylate.

According to a sixteenth embodiment, the personal care composition of according to any preceding embodiment, wherein the water is present in an amount from about 10 to 90 wt. %, based on the total weight of the personal care composition.

According to a seventeenth embodiment, the personal care composition of any preceding embodiment, wherein the polyol is selected from glycerine, 1,3-propanediol, isoprene glycol, pentylene glycol, hexylene glycol, ethylene glycol, propylene glycol, butylene glycol, diethylene glycol, dipropylene glycol, diglycerol, erythritol, arabitol, adonitol, sorbitol, dulcitol, glucose, fructose, xylose, trehalose, sucrose, maltose, saccharose and lactose, and a combination of two or more thereof.

According to an eighteenth embodiment, the personal care composition of any preceding embodiment, wherein the polyol is butylene glycol.

According to a nineteenth embodiment, the personal care composition of any preceding embodiment further comprising a polymeric thickener.

According to a twentieth embodiment, the personal care composition of any preceding embodiment, wherein the composition comprises:
butylene glycol;
   wherein the surfactant system comprises:
an amphoteric surfactant, wherein the amphoteric surfactant comprises cocamidopropyl hydroxysultaine;
an alkyl glucoside, wherein the alkyl glucoside comprises a combination of decyl glucoside and caprylyl/capryl glucoside;
a non-ionic surfactant, wherein the non-ionic surfactant is a blend of decyl glucoside and sodium lauroyl lactylate;
an anionic surfactant, wherein the anionic surfactant comprises sodium cocoyl apple amino acids;
glycerin; and
polyacrylate crosspolymer-6.

According to a twenty-first embodiment, the personal care composition of any preceding embodiment, wherein the personal composition is a rinse off composition.

According to a twenty-second embodiment, the personal care composition of any preceding embodiment, wherein the personal composition is in the form selected from liquid soap, liquid hand soap, a facial cleanser and/or facial soap, shower gel, body wash, and shampoo.

In accordance with a twenty-third embodiment, provided is a personal care composition, comprising:
a polyol;
a surfactant system comprising:
   an anionic surfactant selected from an anionic derivatives of proteins of plant origin, anionic derivatives of proteins of silk protein, a combination of two or more thereof,
   an amphoteric surfactant, and
   a non-ionic surfactant selected from an alkyl glucoside, a lactylate having carbon chains between C₈ and C₁₆, and a combination of two or more thereof;
optionally, water.

According to a twenty-fourth embodiment, the personal care composition of the twenty-third embodiment, wherein the anionic surfactant is selected from potassium lauroyl wheat amino acid, sodium lauroyl wheat amino acid, sodium lauroyl oat amino acid, sodium cocoyl apple amino acid, potassium undecylenoyl hydrolyzed soy protein, sodium undecylenoyl hydrolyzed soy protein, sodium dilauramidoglutamide lysine, sodium cocoyl threoninate, sodium cocoyl glutamate, disodium cocoyl glutamate, and a combination of two or more thereof.

According to a twenty-fifth embodiment, the personal care composition of the twenty-third or twenty-fourth embodiment, wherein the anionic surfactant comprises potassium lauroyl wheat amino acid, sodium lauroyl wheat amino acid, sodium lauroyl oat amino acid, sodium cocoyl apple amino acid, potassium undecylenoyl hydrolyzed soy protein, sodium undecylenoyl hydrolyzed soy protein, sodium dilauramidoglutamide lysine, or a combination of two or more thereof.

According to a twenty-sixth embodiment, the personal care composition of one of the twenty-third to the twenty-fifth embodiment, wherein the amphoteric surfactant comprises a betaine surfactant, sultaine surfactant, or a combination of two or more thereof.

According to a twenty-seventh embodiment, the personal care composition of the twenty-sixth, wherein the amphoteric surfactant comprises a betaine surfactant selected from coca betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, and combinations of two or more thereof.

According to a twenty-eight embodiment, the personal care composition of the twenty-seventh, wherein the betaine surfactant is selected from coco betaine, cocamidopropyl betaine, behenyl betaine, capryl/capramidopropyl betaine, and lauryl betaine, and a combination of two or more thereof.

According to a twenty-ninth embodiment, the personal care composition of one of the twenty-sixth to the twenty-eight embodiment, wherein the amphoteric surfactant comprises a sultaine surfactant is selected from lauramidopropyl hydroxysultaine, cocamidopropyl hydroxysultaine, oleamidopropyl hydroxysultaine, tallowamidopropyl hydroxysultaine, erucamidopropyl hydroxysultaine, lauryl hydroxysultaine, lauramidopropyl hydroxysultaine, and a combination of two or more thereof.

According to a thirtieth embodiment, the personal care composition of one of the twenty-third to the twenty-ninth embodiment, wherein the nonionic surfactant comprises an alkyl glucoside.

According to a thirty-first embodiment, the personal care composition of the thirtieth embodiment, wherein the alkyl glucoside is selected from decyl glucoside, caprylyl/capryl glucoside, caprylyl glucoside, lauryl glucoside, coco-glucoside, octyl glucoside, cetearyl glucoside, cetyl glucoside, hexadecyl glucoside, arachidyl glucoside, and a combination of two or more thereof.

According to a thirty-second embodiment, the personal care composition of one of the twenty-third to the thirty-first embodiment, wherein the nonionic surfactant comprises a lactylate selected from sodium decanoyl/dodecanoyl lactylate, tea-lauroyl lactylate, sodium hexanoyl lactylate, sodium behenoyl lactylate, sodium oleoyl lactylate, sodium decanoyl lactylate, sodium cocoyl lactylate, sodium lauroyl lactylate, sodium isostearoyl lactylate, calcium stearoyl lactylate, sodium stearoyl lactylate, and a combination of two or more thereof.

According to a thirty-third embodiment, the personal care composition of one of the twenty-third to the thirty-second embodiment wherein the nonionic surfactant comprises an alkyl glucoside and a lactylate.

### EXAMPLES

### Example 1

The following are non-limiting examples of facial cleansers of the disclosure comprising the surfactant system.

TABLE 1

| | Formula A | | Formula B | |
|---|---|---|---|---|
| | (Wt. %) | | (Wt. %) | |
| Water | q.s. (e.g., about | | q.s. (e.g., about | |
| | 70% by wt.) | | 70% by wt.) | |
| Preservative(s) | 0.1-1% | (e.g., 0.3) | 0.1-1% | (e.g., 0.5) |
| Glycine, L-Arginine, and | — | | 0.1-3% | |
| Taurine | | | | |
| Polymeric thickener | 0.5-5% | (e.g., 2.0) | 0.5-5% | (e.g., 2.0) |
| Butylene Glycol | 0.5-5% | (e.g., 2.0) | 0.5-5% | (e.g., 2.0) |
| Humectant | 0.5-5% | (e.g., 3.0) | 0.5-5% | (e.g., 3.0) |
| Cocamidopropyl | 1-10% | (e.g., 5.0) | 1-10% | (e.g., 5.0) |
| Hydroxysultaine | | | | |
| Decyl Glucoside | 1-10% | (e.g., 5.0) | 1-10% | (e.g., 5.0) |
| Caprylyl Glucoside | 1-5% | (e.g., 3.0) | 1-5% | (e.g., 3.0) |
| Sodium Cocoyl Apple Amino | 1-10% | (e.g., 5.0) | 1-10% | (e.g., 5.0) |
| Acid Surfactant | | | | |
| Decyl Glucoside (and) | 1-5% | (e.g., 2.0) | 1-5% | (e.g., 2.0) |
| Sodium | | | | |
| Lauroyl Lactylate | | | | |
| Oil | 0.1-1% | (e.g., 0.3) | 0.1-1% | (e.g., 0.3) |
| pH Modifying Agent | 0.1-1% | (e.g., 0.5) | 0.1-1% | (e.g., 0.5) |

| | | |
|---|---|---|
| Total | 100 | 100 |

### Example 2

Two non-limiting, example personal care compositions (Example Compositions C and D) was prepared in accordance with aspects of the disclosure. The formulations of Example Compositions C and D are shown in Table 2.

TABLE 2

| | Ex. C | Ex. D |
|---|---|---|
| INCI Compound Name | (wt. %) | (wt. %) |
| Cocamidopropyl | 2 | 2 |
| Hydroxysultaine | | |
| Sodium N-Coconut Acid-N- | ~1.7 | ~1.7 |
| Methyl Taurate | | |
| Decyl Glucoside | 4 | 4 |
| Sodium Cocoyl Glycinate | 3 | 3 |
| Sodium Cocoyl Apple Amino | 4 | 4 |
| Acids | | |
| Decyl Glucoside & Sodium | 0.5 | 0.5 |
| Layroyl Lactylate | | |
| Glycerin | 3 | 3 |
| Butylene Glycol | | 2 |
| Glycine, Biotive L-Arginine, | 2 | 2 |
| and Taurine | | |
| Saccharide Isomerate | 0-0.25 | |
| Bromelain Enzyme, Pumpkin | 0.4 | |
| Enzyme, Zingiber Officinale | | |
| (Ginger) Root Extract, and | | |
| Bisabolol | | |
| Asiaticoside, Tocopherol, | 0.1 | 0.4 |
| Vitamin E acetate, and/or | | |
| Vitamin C | | |
| Citric Acid and/or sodium | ~0.5 | ~0.5 |
| citrate | | |
| Preservative(s) | 1 | 1 |
| Water | Q.S. to 100 | Q.S. to 100 |

### Example 3

Example Compositions C and D were evaluated to assess their respective rheological foaming characteristics. Samples from Example Compositions C and D were dispensed individually in the form of foam onto the bottom plate of a AR2000 rheometer, available from TA INSTRUMENTS, to separately assess the rheological foaming characteristic of Example Compositions C and D. Parallel plate geometry was used, but with a serrated surface, which prevents slip of the bubbles on the surface of the plate. The temperature of the bottom plate was set to 25° C. After the samples of Example Compositions C or D were pumped in the form of foam onto the bottom plate, the top plate was lowered down until the gap between the two plates became 1 mm. As the gap decreased, the foam was squeezed out.

A strain sweep test was conducted where shear strain ramped from 0.1 to 1000% step-wise with a fixed angular frequency of 10 rad/s. During the measurement, shear stress and elastic modulus were measured.

The elastic modulus was plotted against shear stress in log-log scale. When low shear stress is applied, as the structure of foam was not destroyed, elasticity of the foam can be measured. Elastic modulus is independent from the applied shear stress, which appears as the plateau of elastic modulus (G′). As shear stress increases beyond yield stress, the structure of foam breaks, which results in the decrease in elastic modulus. These two parameters are governed by foam structure. Both parameters typically increase with higher bubble fraction and smaller bubble size, e.g., when foam is denser. G′ and yield stress are obtained by drawing two tangential lines. By extrapolating the fit line from the low stress regime, G′ was obtained. Yield stress is the stress where the two lines meet.

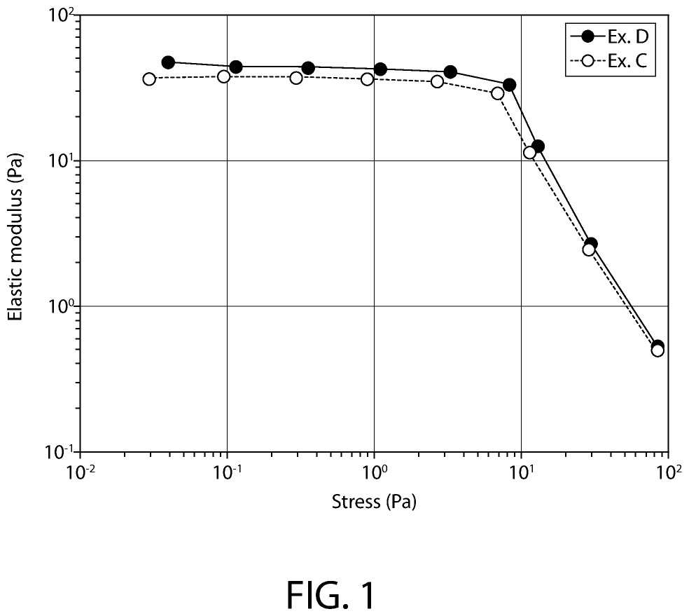

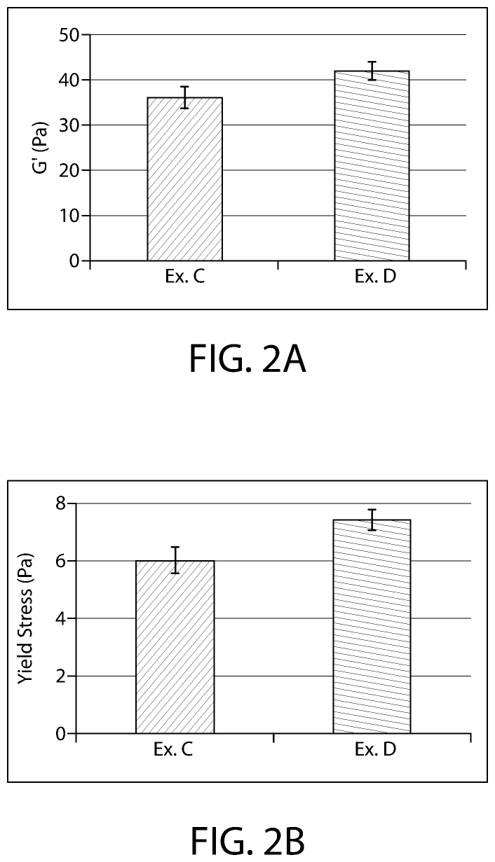

FIG. 1 is a graph of the average rheological foam curves for Example Compositions C and D. FIG. 2A and FIG. 2B are bar graphs of the elasticity and yield stress, respectively, of the foams of Example Compositions C or D.

### Example 4

Example Compositions C and D were evaluated to assess their ability to remove sebum. To evaluate the cleansing ability of Example Compositions C and D, sebum was evenly spread onto films using a spatula. The weight of the sebum after spreading was then measured to make sure that the weight on each film was approximately 0.1 grams. Samples of 0.25 grams of either Example Composition C or Example Composition D were placed on the respective films and gently spread using fingertips. The films were subsequently wet with 0.25 mL of water. After 1 hour, the films were rinsed with water for 40 seconds.

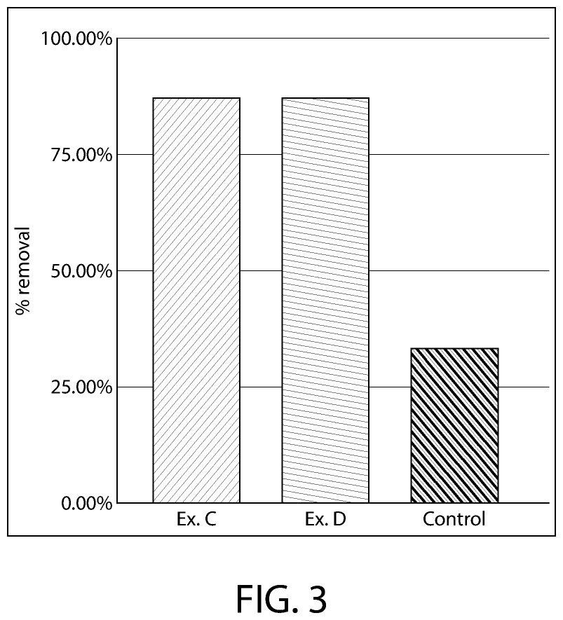

The amount of residual sebum on the films was then measured and analyzed using image J software. FIG. 3 is a bar graph illustrating the amount of sebum removed by Example Composition C, Example Composition D, and water, which was used as a control. Notably, Example Compositions C and D removed about 88% and about 87%, respectively of the sebum while water removed about 34% of the sebum.

The present disclosure has been described with reference to exemplary embodiments. Although a limited number of embodiments have been shown and described, it will be appreciated by those skilled in the art that changes may be made in these embodiments without departing from the principles and spirit of the preceding detailed description. It is intended that the present disclosure be construed as including all such modifications and alterations insofar as they come within the scope of the appended claims or the equivalents thereof.

1. A personal care composition, comprising:
a polyol;
a surfactant system, wherein the surfactant system comprises:
one or more alkyl glucoside(s),
an amphoteric surfactant,
an anionic surfactant selected from an anionic derivatives of proteins of plant origin, anionic derivatives of proteins of silk protein, a combination of two or more thereof, and
a non-ionic surfactant; and
water.

2. The personal care composition of claim 1, wherein the amphoteric surfactant is a sultaine surfactant selected from the group consisting of lauramidopropyl hydroxysultaine, cocamidopropyl hydroxysultaine, oleamidopropyl hydroxysultaine, tallowamidopropyl hydroxysultaine, erucamidopropyl hydroxysultaine, lauryl hydroxysultaine, lauramidopropyl hydroxysultaine, and a combination of two or more thereof.

3. (canceled)

4. The personal care composition of claim 2, wherein the sultaine surfactant comprises cocamidopropyl hydroxysultaine.

5. The personal care composition of claim 1, wherein the alkyl glucoside is C₆₋₂₅ alkyl glucoside; and
wherein the alkyl glucoside is selected from decyl glucoside, caprylyl/capryl glucoside, lauryl glucoside, coco-glucoside, octyl glucoside, cetearyl glucoside, cetyl glucoside, hexadecyl glucoside, arachidyl glucoside, and a combination of two more thereof.

6. (canceled)

7. The personal care composition of claim 1, wherein the alkyl glucoside comprises a combination of decyl glucoside and caprylyl/capryl glucoside.

8. The personal care composition of claim 1, wherein the anionic surfactant is an anionic derivative of a protein hydrolysate of plant origin;
wherein the protein hydrolysate comprises a hydrophobic group; and
wherein the protein hydrolysate is an apple, a wheat, a soybean, or an oat protein hydrolysate comprising an alkyl chain having from 10 to 22 carbon atoms, and salts thereof.

9. (canceled)

10. The personal care composition of claim 8, wherein the protein hydrolysate is an apple protein hydrolysate comprising an alkyl chain having from 10 to 22 carbon atoms.

11. The personal care composition of claim 10, wherein the anionic surfactant comprises sodium cocoyl apple amino acids.

12. The personal care composition of claim 1, wherein the non-ionic surfactant comprises a blend of an alkyl glucoside and a lactylate having carbon chains between C₈ and C₁₆ and
wherein the lactylate is selected from the group consisting of ammonium, sodium, potassium and alkanolamine (e.g., triethanolamine) salts of lauroyl lactylate, cocoyl lactylate, lauroyl lactylate, caproyl lactylate, and a combination of two or more thereof.

13. The personal care composition of claim 12, wherein the alkyl glucoside comprises decyl glucoside.

14. The personal care composition of claim 12, wherein the lactylate comprises sodium lauroyl lactylate.

15. The personal care composition of claim 12, wherein the non-ionic surfactant is a blend of an alkyl glucoside and a lactylate having carbon chains between C₈ and C₁₆, and wherein the surfactant is a blend of decyl glucoside and sodium lauroyl lactylate.

16. (canceled)

17. The personal care composition of claim 1, wherein the polyol is selected from glycerine, 1,3-propanediol, isoprene glycol, pentylene glycol, hexylene glycol, ethylene glycol, propylene glycol, butylene glycol, diethylene glycol, dipropylene glycol, diglycerol, erythritol, arabitol, adonitol, sorbitol, dulcitol, glucose, fructose, xylose, trehalose, sucrose, maltose, saccharose and lactose, and a combination of two or more thereof.

18. The personal care composition of claim 1, wherein the polyol is butylene glycol.

19. The personal care composition of claim 1, further comprising a polymeric thickener.

20. The personal care composition of claim 1, wherein the composition comprises:
butylene glycol;
wherein the surfactant system comprises:
an amphoteric surfactant, wherein the amphoteric surfactant comprises cocamidopropyl hydroxysultaine;
an alkyl glucoside, wherein the alkyl glucoside comprises a combination of decyl glucoside and caprylyl/capryl glucoside;
a non-ionic surfactant, wherein the non-ionic surfactant is a blend of decyl glucoside and sodium lauroyl lactylate;
an anionic surfactant, wherein the anionic surfactant comprises sodium cocoyl apple amino acids;
glycerin; and
polyacrylate crosspolymer-6.

21. The personal care composition of claim 1, wherein the personal composition is a rinse off composition.

22. The personal care composition of claim 1, wherein the personal composition is in the form selected from liquid soap, liquid hand soap, a facial cleanser and/or facial soap, shower gel, body wash, and shampoo.

23. A personal care composition, comprising:
a polyol;
a surfactant system comprising:
an anionic surfactant selected from anionic derivatives of proteins of plant origin, anionic derivatives of proteins of silk protein, and a combination of two or more thereof;
an amphoteric surfactant; and
a non-ionic surfactant selected from an alkyl glucoside, a lactylate having carbon chains between C₈ and C₁₆, and a combination of two or more thereof; and
optionally, water.

24. The personal care composition of claim 23, wherein the anionic surfactant is selected from potassium lauroyl wheat amino acid, sodium lauroyl wheat amino acid, sodium lauroyl oat amino acid, sodium cocoyl apple amino acid, potassium undecylenoyl hydrolyzed soy protein, sodium undecylenoyl hydrolyzed soy protein, sodium dilauramidoglutamide lysine, sodium cocoyl threoninate, sodium cocoyl glutamate, disodium cocoyl glutamate, and a combination of two or more thereof.

25. (canceled)

26. The personal care composition of claim 23, wherein the amphoteric surfactant comprises a betaine surfactant, sultaine surfactant, or a combination of two or more thereof.

27. The personal care composition of claim 26, wherein the amphoteric surfactant comprises a betaine surfactant, and wherein the betaine surfactant is selected from coca betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, and combinations of two or more thereof.

28. The personal care composition of claim 27, wherein the betaine surfactant is selected from coco betaine, cocamidopropyl betaine, behenyl betaine, capryl/capramidopropyl betaine, lauryl betaine, and a combination of two or more thereof.

29. The personal care composition of claim 26, wherein the amphoteric surfactant comprises a sultaine surfactant, and wherein the sultaine surfactant is selected from lauramidopropyl hydroxysultaine, cocamidopropyl hydroxysultaine, oleamidopropyl hydroxysultaine, tallowamidopropyl hydroxysultaine, erucamidopropyl hydroxysultaine, lauryl hydroxysultaine, lauramidopropyl hydroxysultaine, and a combination of two or more thereof.

30. The personal care composition of claim 23, wherein the nonionic surfactant comprises an alkyl glucoside.

31. The personal care composition of claim 30, wherein the alkyl glucoside is selected from decyl glucoside, caprylyl/capryl glucoside, caprylyl glucoside, lauryl glucoside, coco-glucoside, octyl glucoside, cetearyl glucoside, cetyl glucoside, hexadecyl glucoside, arachidyl glucoside, and a combination of two or more thereof.

32. The personal care composition of one of claim 23, wherein the nonionic surfactant comprises a lactylate, and wherein the lactylate is selected from sodium decanoyl/dodecanoyl lactylate, tea-lauroyl lactylate, sodium hexanoyl lactylate, sodium behenoyl lactylate, sodium oleoyl lactylate, sodium decanoyl lactylate, sodium cocoyl lactylate, sodium lauroyl lactylate, sodium isostearoyl lactylate, calcium stearoyl lactylate, sodium stearoyl lactylate, and a combination of two or more thereof.

33. The personal care composition of claim 23, wherein the nonionic surfactant comprises an alkyl glucoside and a lactylate.

34. The personal care composition of claim 23, wherein the personal care composition produces a foam having an elastic modulus of about 10 to about 50 Pa at a stress of at least 8 Pa.

35. The personal care composition of claim 23, wherein the personal care composition produces a foam having an elasticity of about 25 to 50 Pa, as assessed by an ANOVA test.

36. The personal care composition of claim 23, wherein the personal care composition produces a foam having a yield stress of about 4 to about 10 Pa, as assessed by an ANOVA test.

37. A personal care product comprising:
a container comprising a mechanical pump; and
a personal care composition disposed in the container, the personal care composition comprising:
a polyol;
a surfactant system comprising:
an anionic surfactant selected from anionic derivatives of proteins of plant origin, anionic derivatives of proteins of silk protein, and a combination of two or more thereof;
an amphoteric surfactant; and
a non-ionic surfactant selected from an alkyl glucoside, a lactylate having carbon chains between C₈ and C₁₆, and a combination of two or more thereof; and
optionally, water.

38. The personal care product of claim 37, wherein the mechanical pump comprises a foaming chamber and a mesh disposed in the foaming chamber.

39. The personal care product of claim 38, wherein the foaming chamber comprises an inlet and a foam outlet, wherein the cross-sectional area of the foam outlet is greater than the cross-sectional area of the inlet by about 100 to about 500%.

40. The personal care product of claim 38, wherein the mesh is configured to be a mesh screen or a mesh ball.

41. The personal care product of claim 37, wherein the mechanical pump is configured to promote mixing of the personal care composition with air to promote the production of foam.

42. The personal care product of claim 37, wherein the personal care composition is adapted to produce, when dispensed using the mechanical pump, a foam having an elastic modulus of about 10 to about 50 Pa at a stress of at least 8 Pa.

43. The personal care product of claim 37, wherein the personal care composition is adapted to produce, when dispensed using the mechanical pump, a foam having an elasticity of about 25 to 50 Pa.

44. The personal care product of claim 37, wherein the personal care composition is adapted to produce, when dispensed using the mechanical pump, a foam having a yield stress of about 4 to about 10 Pa.
